(19) United States
(12) Patent Application Publication
(10) Pub. No.: US 2010/0273770 A1
(43) Pub. Date: Oct. 28, 2010

(54) 1, 2, 4 -OXADIAZOLE COMPOUNDS FOR THE TREATMENT OF AUTOIMMUNE DISEASES

(76) Inventors: Jag Paul Heer, Essex (GB); Thomas Daniel Heightman, Essex (GB); David Nigel Hurst, Essex (GB); Christopher Norbert Johnson, Essex (GB); John Skidmore, Essex (GB); Ian David Wall, Essex (GB)

Correspondence Address: GlaxoSmithKline;GLOBAL PATENTS -US, UW2220, P. O. BOX 1539, KING OF PRUSSIA, PA, 19406-0939, US

(21) Appl. No.: 12/747,198

(22) PCT Filed: Dec. 19, 2008
(86) PCT No.: PCT/EP08/67972

§ 371 Date: Jun. 10, 2010

(30) Foreign Application Priority Data
Dec. 21, 2007 (GB) 0725102.8

(51) Int. Cl. A61K 31/55; C07D 413/10; C07D 413/14; A61K 31/553

(52) U.S. Cl. 514/211.09; 540/593; 540/552; 514/213.01

(57) ABSTRACT

The present invention relates to novel oxadiazole derivatives having pharmacological activity, processes for their preparation, pharmaceutical compositions containing them and their use in the treatment of various disorders.

The present invention relates to novel oxadiazole derivatives having pharmacological activity, processes for their preparation, pharmaceutical compositions containing them and their use in the treatment of various disorders.

Sphingosine 1-phosphate (S1P) is a bioactive lipid mediator formed by the phosphorylation of sphingosine by sphingosine kinases and is found in high levels in the blood. It is produced and secreted by a number of cell types, including those of hematopoietic origin such as platelets and mast cells (Okamoto et al 1998 J Biol Chem 273(42):27104; Sanchez and Hla 2004, J Cell Biochem 92:913). It has a wide range of biological actions, including regulation of cell proliferation, differentiation, motility, vascularisation, and activation of inflammatory cells and platelets (Pyne and Pyne 2000, Biochem J. 349: 385). Five subtypes of S1P responsive receptor have been described, S1P1 (Edg-1), S1P2 (Edg-5), S1P3 (Edg-3), S1P4 (Edg-6), and S1P5 (Edg-8), forming part of the G-protein coupled endothelial differentiation gene family of receptors (Chun et al 2002 Pharmacological Reviews 54:265, Sanchez and Hla 2004 J Cellular Biochemistry, 92:913). These 5 receptors show differential mRNA expression, with S1P1-3 being widely expressed, S1P4 expressed on lymphoid and hematopoietic tissues and S1P5 primarily in brain and to a lower degree in spleen. They signal via different subsets of G proteins to promote a variety of biological responses (Kluk and Hla 2002 Biochem et Biophysica Acta 1582:72, Sanchez and Hla 2004, J Cellular Biochem 92:913).

Proposed roles for the S1P1 receptor include lymphocyte trafficking, cytokine induction/suppression and effects on endothelial cells (Rosen and Goetzl 2005 Nat Rev Immunol. 5:560). Agonists of the S1P1 receptor have been used in a number of autoimmune and transplantation animal models, including Experimental Autoimmune Encephalomelitis (EAE) models of MS, to reduce the severity of the induced disease (Brinkman et al 2003 JBC 277:21453; Fujino et al 2003 J Pharmacol Exp Ther 305:70; Webb et al 2004 J Neuroimmunol 153:108; Rausch et al 2004 J Magn Reson Imaging 20:16). This activity is reported to be mediated by the effect of S1P1 agonists on lymphocyte circulation through the lymph system. Treatment with S1P1 agonists results in the sequestration of lymphocytes within secondary lymphoid organs such as the lymph nodes, inducing a reversible peripheral lymphopoenia in animal models (Chiba et al 1998, J Immunology 160:5037, Forrest et al 2004 J Pharmacol Exp Ther 309:758; Sanna et al 2004 JBC 279:13839). Published data on agonists suggests that compound treatment induces loss of the S1P1 receptor from the cell surface via internalisation (Graler and Goetzl 2004 FASEB J 18:551; Matloubian et al 2004 Nature 427:355; Jo et al 2005 Chem Biol 12:703) and it is this reduction of S1P1 receptor on immune cells which contributes to the reduction of movement of T cells from the lymph nodes back into the blood stream.

S1P1 gene deletion causes embryonic lethality. Experiments to examine the role of the S1P1 receptor in lymphocyte migration and trafficking have included the adoptive transfer of labelled S1P1 deficient T cells into irradiated wild type mice. These cells showed a reduced egress from secondary lymphoid organs (Matloubian et al 2004 Nature 427:355).

S1P1 has also been ascribed a role in endothelial cell junction modulation (Allende et al 2003 102:3665, Blood Singelton et al 2005 FASEB J 19:1646). With respect to this endothelial action, S1P1 agonists have been reported to have an effect on isolated lymph nodes which may be contributing to a role in modulating immune disorders. S1P1 agonists caused a closing of the endothelial stromal ‘gates’ of lymphatic sinuses which drain the lymph nodes and prevent lymphocyte egress (Wei wt al 2005, Nat. Immunology 6:1228).

The immunosuppressive compound FTY720 (JP11080026-A) has been shown to reduce circulating lymphocytes in animals and man, have disease modulating activity in animal models of immune disorders and reduce remission rates in relapsing remitting Multiple Sclerosis (Brinkman et al 2002 JBC 277:21453, Mandala et al 2002 Science 296:346, Fujino et al 2003 J Pharmacology and Experimental Therapeutics 305:45658, Brinkman et al 2004 American J Transplantation 4:1019, Webb et al 2004 J Neuroimmunology 153:108, Morris et al 2005 Eur J Immunol 35:3570, Chiba 2005 Pharmacology and Therapeutics 108:308, Kahan et al 2003, Transplantation 76:1079, Kappos et al 2006 New Eng J Medicine 335:1124). This compound is a prodrug that is phosphorylated in vivo by sphingosine kinases to give a molecule that has agonist activity at the S1P1, S1P3, 51P4 and S1P5 receptors. Clinical studies have demonstrated that treatment with FTY720 results in bradycardia in the first 24 hours of treatment (Kappos et al 2006 New Eng J Medicine 335:1124). The bradycardia is thought to be due to agonism at the S1P3 receptor, based on a number of cell based and animal experiments. These include the use of S1P3 knock-out animals which, unlike wild type mice, do not demonstrate bradycardia following FTY720 administration and the use of S1P1 selective compounds. (Hale et al 2004 Bioorganic & Medicinal Chemistry Letters 14:3501, Sanna et al 2004 JBC 279:13839, Koyrakh et al 2005 American J Transplantation 5:529)

Hence, there is a need for S1P1 receptor agonist compounds with selectivity over S1P3 which might be expected to show a reduced tendency to induce bradycardia.

The following patent applications describe oxadiazole derivatives as S1P1 agonists: WO03/105771, WO05/058848, WO06/047195, WO06/100633, WO06/115188, WO06/131336, WO07/024,922 and WO07/116,866.

The following patent applications describe tetrahydroisoquinolinyl-oxadiazole derivatives as S1P receptor agonists: WO06/064757, WO06/001463, WO04/113330.

WO08/064,377 describes benzocycloheptyl analogs having S1P1 receptor activity.

A structurally novel class of compounds has now been found which provides agonists of the S1P1 receptor.

The present invention therefore provides compounds of formula (I) or a pharmaceutically acceptable salt thereof thereof:

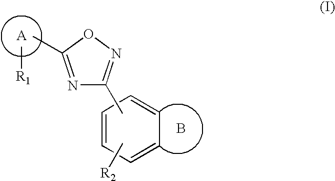

A is phenyl or a 5 or 6-membered heteroaryl ring;
R₁ is up to two substituents independently selected from halogen, C₍₁₋₃₎alkoxy, C₍₁₋₃₎fluoroalkyl, cyano, optionally substituted phenyl, C₍₁₋₃₎fluoroalkoxy, C₍₁₋₆₎alkyl and C₍₃₋₆₎cycloalkyl;
R₂ is hydrogen, halogen or C₍₁₋₄₎alkyl;
B is a 7 membered saturated ring selected from the following:

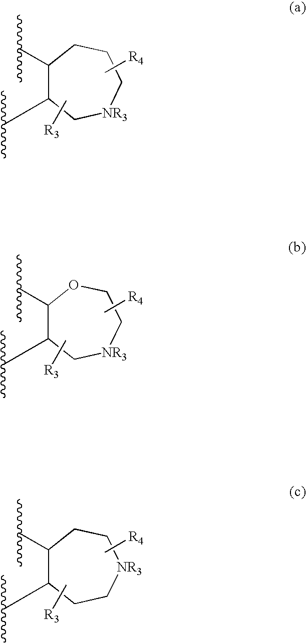

R₃ is hydrogen or C₍₁₋₃₎alkyl optionally substituted by oxygen;

### R₄ is (CH₂)₁₋₃CONH₂, (CH₂)₁₋₃OH, CO₂H or (CH₂)₁₋₃CO₂H.

In one embodiment of the invention,

A is phenyl; and/or
R₁ is up to two substituents independently selected from chloro and isopropoxy;
and/or
R₂ is hydrogen; and/or
B is (a) or (b); and/or
R₃ is hydrogen; and/or

### R₄ is (CH₂)₂CONH₂, (CH₂)₁₋₃OH, CO₂H or (CH₂)₂CO₂H.

In one embodiment of the invention,

A is phenyl or pyridyl;
R₁ is up to two substituents independently selected from chloro, cyano and isopropoxy;
R₂ is hydrogen;

### B is (a) or (b);

R₃ is hydrogen;

### R₄ is (CH₂)₂CONH₂, (CH₂)₁₋₃OH, CO₂H or (CH₂)₁₋₃CO₂H.

In one embodiment A is phenyl or pyridyl. In another embodiment A is phenyl. In another embodiment A is 3,4-disubstituted phenyl.

In one embodiment R₁ is two substituents one of which is C₍₁₋₃₎alkoxy, the other selected from halogen or cyano. In another embodiment R₁ is two substituents, one of which is isopropoxy and the other is selected from chloro or cyano. In another embodiment R₁ is two substituents selected from chloro, isopropoxy and cyano. In another embodiment R₁ is chloro and isopropoxy. In a further embodiment R₁ is chloro at the 3-position and isopropoxy at the 4-position when A is phenyl or R₁ is chloro at the 5-position and isopropoxy at the 6-position when A is pyridinyl. In another embodiment R₁ is isopropoxy and cyano. In a further embodiment R₁ is cyano at the 3-position and isopropoxy at the 4-position when A is phenyl or R₁ is chloro at the 5-position and isopropoxy at the 6-position when A is pyridinyl.

In one embodiment B is (a) or (b). In another embodiment B is (b).

In one embodiment R₂ is hydrogen.

In one embodiment R₃ is hydrogen.

In one embodiment R₄ is (CH₂)₂CONH₂, (CH₂)₁₋₃OH, CO₂H or (CH₂)₁₋₃CO₂H. In another embodiment (CH₂)₁₋₃CO₂H.

The term “alkyl” as a group or part of a group e.g. alkoxy or hydroxyalkyl refers to a straight or branched alkyl group in all isomeric forms. The term “C₍₁₋₆₎ alkyl” refers to an alkyl group, as defined above, containing at least 1, and at most 6 carbon atoms Examples of such alkyl groups include methyl, ethyl, propyl, iso-propyl, n-butyl, iso-butyl, sec-butyl, or tert-butyl. Examples of such alkoxy groups include methoxy, ethoxy, propoxy, iso-propoxy, butoxy, iso-butoxy, sec-butoxy and tert-butoxy.

Suitable C₍₃₋₆₎cycloalkyl groups include cyclopropyl, cyclobutyl, cyclopentyl and cyclohexyl.

As used herein, the term “halogen” refers to fluorine (F), chlorine (Cl), bromine (Br), or iodine (I) and the term “halo” refers to the halogen: fluoro (—F), chloro (—Cl), bromo (—Br) and iodo (—I).

The term “heteroaryl” represents an unsaturated ring which comprises one or more heteroatoms selected from O, N or S. Examples of 5 or 6 membered heteroaryl rings include pyrrolyl, triazolyl, thiadiazolyl, tetrazolyl, imidazolyl, pyrazolyl, isothiazolyl, thiazolyl, isoxazolyl, oxazolyl, oxadiazolyl, furazanyl, furanyl, thienyl, pyridyl, pyrimidinyl, pyrazinyl, pyridazinyl and triazinyl.

In certain of the compounds of formula (I), dependent upon the nature of the substituent there are chiral carbon atoms and therefore compounds of formula (I) may exist as stereoisomers. The invention extends to all optical isomers such as stereoisomeric forms of the compounds of formula (I) including enantiomers, diastereoisomers and mixtures thereof, such as racemates. The different stereoisomeric forms may be separated or resolved one from the other by conventional methods or any given isomer may be obtained by conventional stereoselective or asymmetric syntheses.

Certain of the compounds herein can exist in various tautomeric forms and it is to be understood that the invention encompasses all such tautomeric forms.

It is understood that certain compounds of the invention contain both acidic and basic groups and may therefore exist as zwitterions at certain pH values.

Suitable compounds of the invention are:
3-[7-(5-{3-chloro-4-[(1-methylethyl)oxy]phenyl}-1,2,4-oxadiazol-3-yl)-2,3,4,5-tetrahydro-1H-2-benzazepin-1-yl]propanamide
3-[7-(5-{3-chloro-4-[(1-methylethy)oxy]phenyl}-1,2,4-oxadiazol-3-yl)-2,3,4,5-tetrahydro-1H-2-benzazepin-1-yl]propanoic acid
3-[7-(5-{3-chloro-4-[(1-methylethyl)oxy]phenyl}-1,2,4-oxadiazol-3-yl)-2,3,4,5-tetrahydro-1H-2-benzazepin-1-yl]-1-propanol
[7-(5-{3-chloro-4-[(1-methylethyl)oxy]phenyl}-1,2,4-oxadiazol-3-yl)-2,3,4,5-tetrahydro-1,4-benzoxazepin-3-yl]methanol
7-(5-{3-chloro-4-[(1-methylethyl)oxy]phenyl}-1,2,4-oxadiazol-3-yl)-2,3,4,5-tetrahydro-1,4-benzoxazepine-3-carboxylic acid
[9-(5-{3-Chloro-4-[(1-methylethyl)oxy]phenyl}-1,2,4-oxadiazol-3-yl)-2,3,4,5-tetrahydro-1,4-benzoxazepin-5-yl]acetic acid
[9-(5-{3-cyano-4-[(1-methylethyl)oxy]phenyl}-1,2,4-oxadiazol-3-yl)-2,3,4,5-tetrahydro-1,4-benzoxazepin-5-yl]acetic acid
[9-(5-{5-Chloro-6-[(1-methylethyl)oxy]-3-pyridinyl}-1,2,4-oxadiazol-3-yl)-2,3,4,5-tetrahydro-1,4-benzoxazepin-5-yl]acetic acid
4-[9-(5-{3-cyano-4-[(1-methylethyl)oxy]phenyl}-1,2,4-oxadiazol-3-yl)-2,3,4,5-tetrahydro-1,4-benzoxazepin-5-yl]butanoic acid
4-[9-(5-{5-chloro-6-[(1-methylethyl)oxy]-3-pyridinyl}-1,2,4-oxadiazol-3-yl)-2,3,4,5-tetrahydro-1,4-benzoxazepin-5-yl]butanoic acid
4-[9-(5-{3-chloro-4-[(1-methylethyl)oxy]phenyl}-1,2,4-oxadiazol-3-yl)-2,3,4,5-tetrahydro-1,4-benzoxazepin-5-yl]butanoic acid
3-[7-(5-{3-chloro-4-[(1-methylethyl)oxy]phenyl}-1,2,4-oxadiazol-3-yl)-2,3,4,5-tetrahydro-1,4-benzoxazepin-3-yl]propanoic acid
   or pharmaceutically acceptable salts thereof.

Pharmaceutically acceptable derivatives of compounds of formula (I) include any pharmaceutically acceptable salt, ester or salt of such ester of a compound of formula (I) which, upon administration to the recipient is capable of providing (directly or indirectly) a compound of formula (I) or an active metabolic or residue thereof.

The compounds of formula (I) can form salts. It will be appreciated that for use in medicine the salts of the compounds of formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. Certain of the compounds of formula (I) may form acid addition salts with one or more equivalents of the acid. The present invention includes within its scope all possible stoichiometric and non-stoichiometric forms.

Salts may also be prepared from pharmaceutically acceptable bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic bases include salts of primary, secondary, and tertiary amines; substituted amines including naturally occurring substituted amines; and cyclic amines. Particular pharmaceutically acceptable organic bases include arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, procaine, purines, theobromine, triethylamine, trimethylamine, tripropylamine, tris(hydroxymethyl)aminomethane (TRIS, trometamol) and the like. Salts may also be formed from basic ion exchange resins, for example polyamine resins. When the compound of the present invention is basic, salts may be prepared from pharmaceutically acceptable acids, including inorganic and organic acids. Such acids include acetic, benzenesulfonic, benzoic, camphorsulfonic, citric, ethanesulfonic, ethanedisulfonic, fumaric, gluconic, glutamic, hydrobromic, hydrochloric, isethionic, lactic, maleic, malic, mandelic, methanesulfonic, mucic, pamoic, pantothenic, phosphoric, propionic, succinic, sulfuric, tartaric, p-toluenesulfonic acid, and the like.

Pharmaceutically acceptable salts may be prepared conventionally by reaction with the appropriate acid or acid derivative. Pharmaceutically acceptable salts with bases may be prepared conventionally by reaction with the appropriate inorganic or organic base.

The compounds of formula (I) may be prepared in crystalline or non-crystalline form, and, if crystalline, may optionally be hydrated or solvated. This invention includes within its scope stoichiometric hydrates or solvates as well as compounds containing variable amounts of water and/or solvent.

Included within the scope of the invention are all salts, solvates, hydrates, complexes, polymorphs, prodrugs, radiolabelled derivatives, stereoisomers and optical isomers of the compounds of formula (I).

In a further aspect, this invention provides processes for the preparation of a compound of formula (I).

The potencies and efficacies of the compounds of this invention for the S1P1 receptor can be determined by GTPγS assay performed on the human cloned receptor as described herein. Compounds of formula (I) have demonstrated agonist activity at the S1P1 receptor, using functional assays described herein.

Compounds of formula (I) and their pharmaceutically acceptable salts are therefore of use in the treatment of conditions or disorders which are mediated via the S1P1 receptor. In particular the compounds of formula (I) and their pharmaceutically acceptable salts are of use in the treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes (herein after referred to as the “Disorders of the Invention”).

Compounds of formula (I) and their pharmaceutically acceptable salts are therefore of use in the treatment of lupus erythematosis.

Compounds of formula (I) and their pharmaceutically acceptable salts are therefore of use in the treatment of psoriasis.

Compounds of formula (I) and their pharmaceutically acceptable salts are therefore of use in the treatment of multiple sclerosis.

It is to be understood that “treatment” as used herein includes prophylaxis as well as alleviation of established symptoms.

Thus the invention also provides compounds of formula (I) or pharmaceutically acceptable salts thereof, for use as therapeutic substances, in particular in the treatment of the conditions or disorders mediated via the S1P1 receptor. In particular the invention provides a compound of formula (I) or a pharmaceutically acceptable salt thereof for use as a therapeutic substance in the treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

Compounds of formula (I) and their pharmaceutically acceptable salts are of use as therapeutic substances in the treatment of lupus erythematosis.

Compounds of formula (I) and their pharmaceutically acceptable salts are of use as therapeutic substances in the treatment of psoriasis.

Compounds of formula (I) and their pharmaceutically acceptable salts are of use as therapeutic substances in the treatment of multiple sclerosis.

The invention further provides a method of treatment of conditions or disorders in mammals including humans which can be mediated via the S1P1 receptor, which comprises administering to the sufferer a therapeutically safe and effective amount of a compound of formula (I) or a pharmaceutically acceptable salt thereof. In particular the invention provides a method of treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes, which comprises administering to the sufferer a therapeutically safe and effective amount of a compound of formula (I) or a pharmaceutically acceptable salt thereof.

The invention provides a method of treatment of lupus erythematosis, which comprises administering to the sufferer a therapeutically safe and effective amount of a compound of formula (I) or a pharmaceutically acceptable salt thereof.

The invention provides a method of treatment of psoriasis, which comprises administering to the sufferer a therapeutically safe and effective amount of a compound of formula (I) or a pharmaceutically acceptable salt thereof.

The invention provides a method of treatment of multiple sclerosis, which comprises administering to the sufferer a therapeutically safe and effective amount of a compound of formula (I) or a pharmaceutically acceptable salt thereof.

In another aspect, the invention provides for the use of a compound of formula (I) or a pharmaceutically acceptable salt thereof in the manufacture of a medicament for use in the treatment of the conditions or disorders mediated via the S1P1 receptor.

In particular the invention provides a compound of formula (I) or a pharmaceutically acceptable salt thereof for use in the manufacture of a medicament for use in the treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

Compounds of formula (I) and their pharmaceutically acceptable salts are of use in the manufacture of a medicament for use in the treatment of lupus erythematosis.

Compounds of formula (I) and their pharmaceutically acceptable salts are of use in the manufacture of a medicament for use in the treatment of psoriasis.

Compounds of formula (I) and their pharmaceutically acceptable salts are of use in the manufacture of a medicament for use in the treatment of multiple sclerosis.

In order to use the compounds of formula (I) and pharmaceutically acceptable salts thereof in therapy, they will normally be formulated into a pharmaceutical composition in accordance with standard pharmaceutical practice. The present invention also provides a pharmaceutical composition, which comprises a compound of formula (I) or a pharmaceutically acceptable salt thereof, and a pharmaceutically acceptable carrier or excipient.

In a further aspect, the present invention provides a process for preparing a pharmaceutical composition, the process comprising mixing a compound of formula (I) or a pharmaceutically acceptable salt thereof and a pharmaceutically acceptable carrier or excipient.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents (e.g. pregelatinised maize starch, polyvinylpyrrolidone or hydroxypropyl methylcellulose); fillers (e.g. lactose, microcrystalline cellulose or calcium hydrogen phosphate); tabletting lubricants (e.g. magnesium stearate, talc or silica); disintegrants (e.g. potato starch or sodium starch glycollate); and acceptable wetting agents (e.g. sodium lauryl sulphate). The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents (e.g. sorbitol syrup, cellulose derivatives or hydrogenated edible fats), emulsifying agents (e.g. lecithin or acacia), non-aqueous vehicles (which may include edible oils e.g. almond oil, oily esters, ethyl alcohol or fractionated vegetable oils), preservatives (e.g. methyl or propyl-p-hydroxybenzoates or sorbic acid), and, if desired, conventional flavourings or colorants, buffer salts and sweetening agents as appropriate. Preparations for oral administration may be suitably formulated to give controlled release of the active compound.

For parenteral administration, fluid unit dosage forms are prepared utilising a compound of the invention or pharmaceutically acceptable salts thereof and a sterile vehicle. Formulations for injection may be presented in unit dosage form e.g. in ampoules or in multi-dose, utilising a compound of the invention or pharmaceutically acceptable derivatives thereof and a sterile vehicle, optionally with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. Advantageously, a surfactant or wetting agent is included in the composition to facilitate uniform distribution of the compound.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compounds of formula (I) or pharmaceutically acceptable salts thereof may also be formulated in rectal compositions such as suppositories or retention enemas, e.g. containing conventional suppository bases such as cocoa butter or other glycerides.

The compounds of formula (I) or pharmaceutically acceptable salts thereof may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compounds of formula (I) or pharmaceutically acceptable salts thereof, may be formulated as solutions for administration via a suitable metered or unitary dose device or alternatively as a powder mix with a suitable carrier for administration using a suitable delivery device. Thus compounds of formula (I) or pharmaceutically acceptable salts thereof may be formulated for oral, buccal, parenteral, topical (including ophthalmic and nasal), depot or rectal administration or in a form suitable for administration by inhalation or insufflation (either through the mouth or nose).

The compounds of formula (I) or pharmaceutically acceptable salts thereof may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

The composition may contain from 0.1% to 99% by weight, preferably from 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, 1.0 to 500 mg or 1.0 to 200 mg and such unit doses may be administered more than once a day, for example two or three times a day.

Compounds of formula (I) or pharmaceutically acceptable salts thereof may be used in combination preparations, in combination with other active ingredients. For example, the compounds of the invention may be used in combination with cyclosporin A, methotrexate, steriods, rapamycin, proinflammatory cytokine inhibitors, immunomodulators including biologicals or other therapeutically active compounds.

The subject invention also includes isotopically-labeled compounds, which are identical to those recited in formulas I and following, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ³H, ¹¹C, ¹⁴C, ¹⁸F, ¹²³I and ¹²⁵I.

Compounds of the present invention and pharmaceutically acceptable saltss of said compounds that contain the aforementioned isotopes and/or other isotopes of other atoms are within the scope of the present invention. Isotopically-labeled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula (I) and following of this invention can generally be prepared by carrying out the procedures disclosed in the Schemes and/or in the Examples below, by substituting a readily available isotopically labelled reagent for a non-isotopically labeled reagent.

In a further aspect, this invention provides processes for preparation of a compound of formula (I).

One route which may be used to prepare compounds of formula (I) when B is

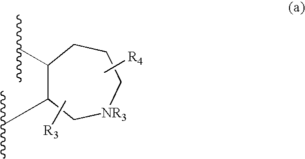

is illustrated in Scheme 1 wherein R₁, R₂ and A are as defined for formula (I) above and the side chain R₄ is located in the 1-position, where n is 1-3, R₂ is hydrogen or C₍₁₋₄₎alkyl, R₃ is hydrogen, R is alkyl (eg. ethyl), hal is chloro, bromo or iodo and P, P₁ are protecting groups,

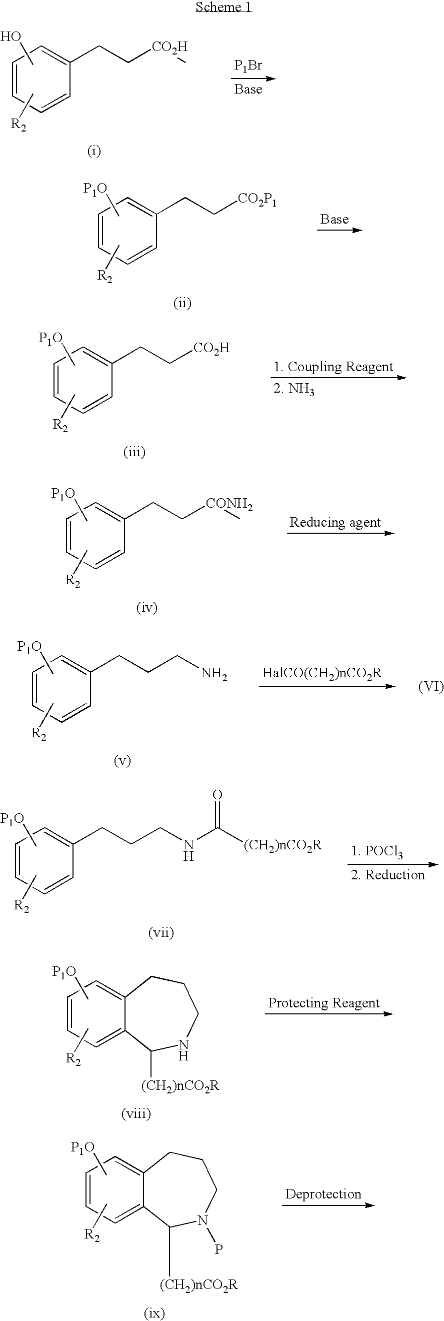

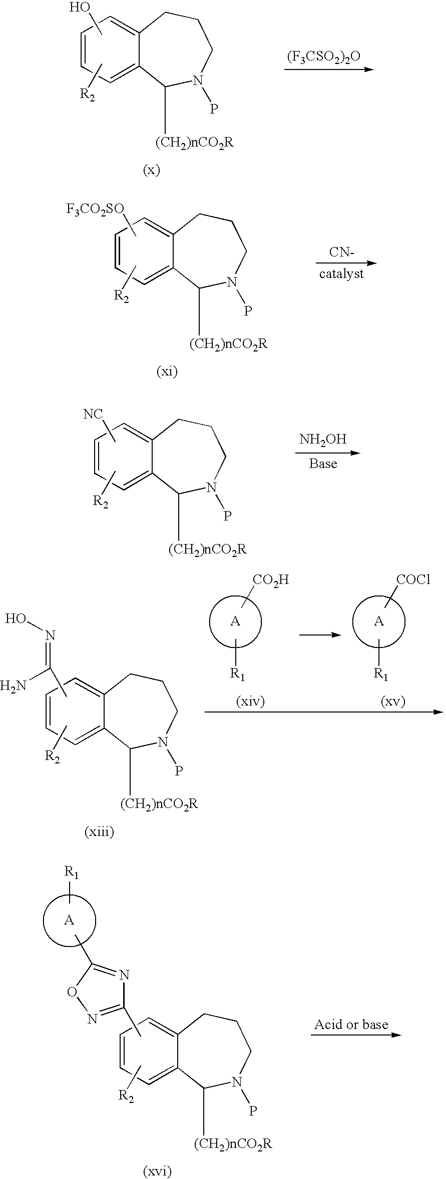

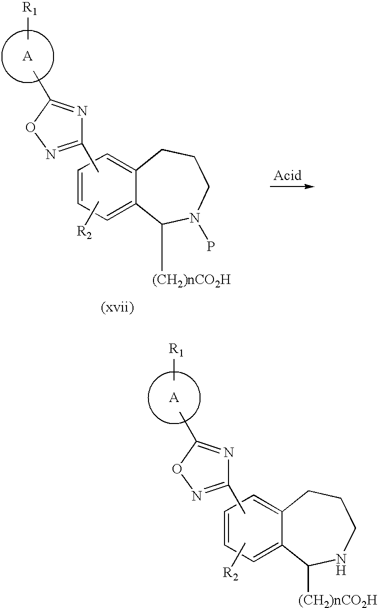

Compounds of formula (I) which are commercially available (e.g. Fluorochem), may be converted into compounds of formula (II), where for example, P₁ is a protecting group such as benzyl, by treatment with an alkylating agent such as benzyl bromide in the presence of a base such as potassium carbonate in a suitable solvent such as DMF. Compounds of formula (II) may be converted into compounds of formula (iii) by hydrolysis using an appropriate base such as aqueous sodium hydroxide in a suitable solvent such as ethanol at an elevated temperature such as 80° C. Compounds of formula (iii) may be converted to compounds of formula (Iv), by conventional means such as treatment with a suitable amide coupling agent such as ethyl chloroformate followed by reaction with ammonia at reduced temperature such as −10° C. and in an appropriate solvent such as THF. Compounds of formula (Iv) may be converted into compounds of formula (v) by treatment with an appropriate reducing agent such as lithium aluminium hydride at a low temperature such as below 15° C., in a solvent such as THF, then elevating the temperature for example to 80° C. Compounds of formula (v) may be converted into compounds of formula (vii) by treatment with an appropriate acyl halide (vi) in the presence of a base such as triethylamine in a solvent such as dichloromethane. The acylating agents (vi) are typically commercially available or may be prepared using standard methods. Compounds of formula (viii) may be converted into compounds of formula (viii) by, for example, treatment with phosphorus oxychloride in a solvent such as acetonitrile at an elevated temperature such as at reflux followed by reduction using a suitable reducing agent such as sodium borohydride in a suitable solvent such as methanol added at a low temperature such as 0° C. and warming to room temperature. Compounds of formula (viii) may be converted to a protected derivative (ix), where P represents a suitable protecting group such as t-butyloxy carbonyl (BOC), for example by treatment with bis(1,1-dimethylethyl) dicarbonate in the presence of a base such as triethylamine in a suitable solvent such as dichloromethane (DCM). Compounds of formula (ix) where P₁ represents a protecting group such as benzyl may be de-protected to give compounds of formula (x) for example by hydrogenolysis using a suitable catalyst such as palladium. Compounds of formula (x) may be converted into compounds of formula (xi) by treatment with a suitable reagent such as trifluoromethanesulphonic anhydride using a suitable base such as pyridine. Compounds of formula (xi) may be converted into compounds of formula (xii) by treatment with a suitable cyanide source such as zinc cyanide in the presence of a catalyst such as tetrakistriphenylphosphine palladium (0) in a suitable solvent such as dimethylformamide (DMF) at an elevated temperature such as 80° C. Compounds of formula (xii) may be converted into compounds of formula (xiii) by treatment with hydroxylamine hydrochloride and an appropriate base, such as sodium bicarbonate, in a solvent such as methanol or ethanol at an elevated temperature such as 60° C. Compounds of formula (xiii) may be converted into compounds of formula (xvi) by treatment with a carboxylic acid chloride of formula (xv) in the presence of a base such as triethylamine in a suitable solvent such as DMF. Such reactions are typically stirred for a period of time at room temperature, then at elevated temperatures, such as 120° C. Acid chlorides of formula (xv) are either commercially available or may be prepared from the corresponding acid (xiv) by conventional means. Alternatively Compounds of formula (xiii) may be converted into compounds of formula (xvi) by treatment with a carboxylic acid of formula (xiv) in the presence of a suitable amide coupling agent such as 1-(3-dimethylaminopropyl)-3-ethylcarbodiimide hydrochloride (EDAC) 1-hydroxybenzotriazole (HOBt) in a suitable solvent such as DMF. Such reactions are typically carried out at elevated temperature, such as 50-80° C. Typically, the acid (xiv), EDAC and HOBt are stirred for a period of time at room temperature prior to addition of the compound of formula (xiii). Compounds of formula (xvi) may be converted into compounds of formula (xvii) by treatment with a base such as aqueous sodium hydroxide in an alcoholic solvent such as ethanol or methanol. Compounds of formula (xvii) where P represents a suitable protecting group such as t-butyloxy carbonyl (BOC), may be converted into certain compounds of formula (I) by treatment with a suitable acid, typically trifluoroacetic acid or hydrochloric acid.

One route which may be used to prepare compounds of formula (I) wherein B is

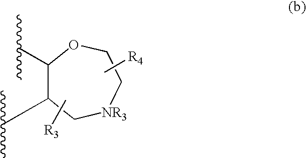

is illustrated in Scheme 2 wherein R₁, R₂ and A are as defined for formula (I) and the side chain R₄ is located in the 5-position of the benzoxazepine ring, R₃ is hydrogen, R₄ is (CH₂)₂CO₂H, R is alkyl (e.g. methyl) and P is a protecting group.

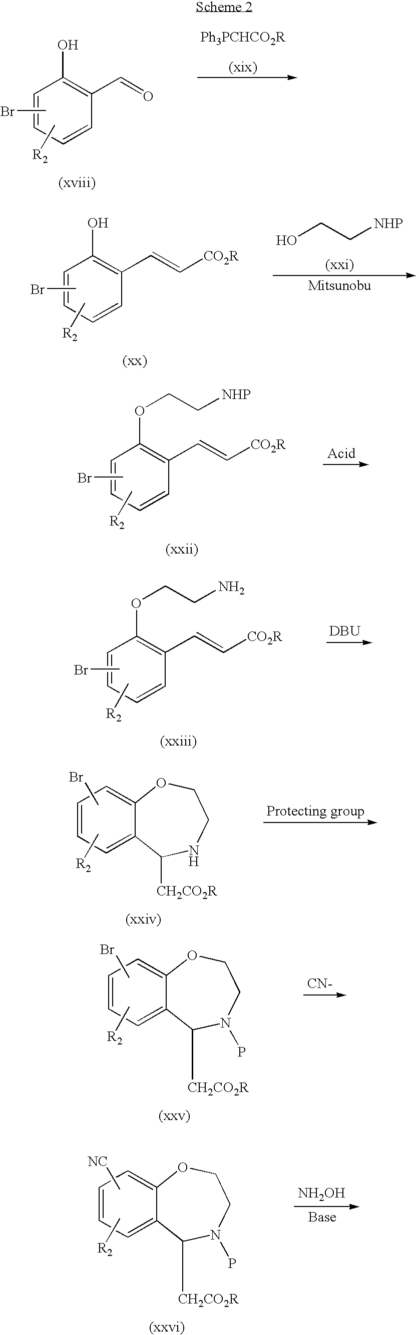

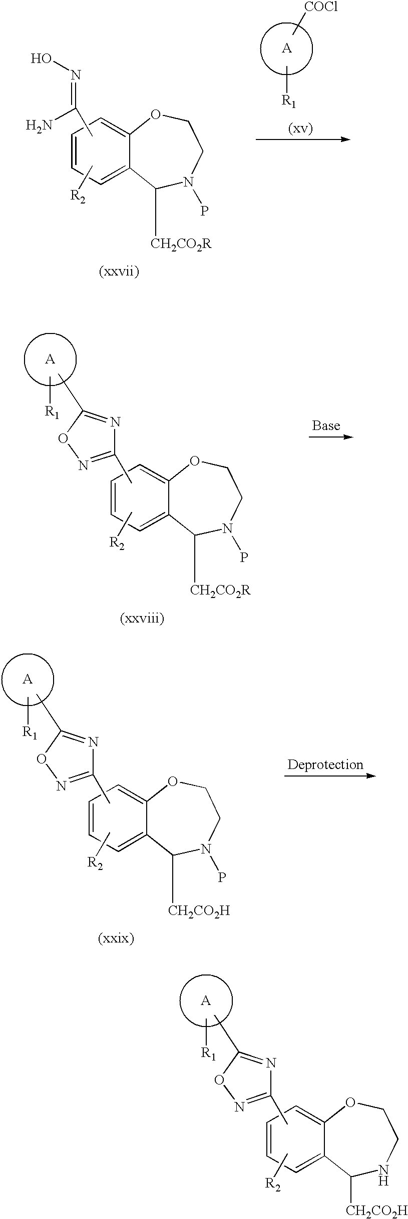

Compounds of formula (xviii) which are commercially available (e.g. Aldrich), may be converted into compounds of formula (xx), for example by treatment with a Wittig reagent (xix) such as (carbethoxymethylene)triphenylphosphorane in a suitable solvent such as dichloromethane. Compounds of formula (xx) may be converted into compounds of formula (xxii) by treatment with a suitable N-protected (such as Boc) ethanolamine (xxi) under Mitsunobu conditions using, for example diisopropyl azodicarboxylate (DIAD) and triphenylphosphine in a suitable solvent such as THF. Compounds of formula (xxii) where P represents a suitable protecting group such as t-butyloxy carbonyl (BOC), may be converted into compounds of formula (xxiii) by treatment with an acid, typically trifluoroacetic acid or hydrochloric acid. Compounds of formula (xxiii) may be converted into compounds of formula (xxiv) by treatment with a suitable reagent such as 1,8-diazabicyclo[5.4.0]undec-7-ene (DBU) in a suitable solvent such as THF. Compounds of formula (xxiv) may be converted into a suitable N-protected derivative (xxv) where P represents a suitable protecting group such as t-butyloxy carbonyl (BOC), for example by treatment with bis(1,1-dimethylethyl) dicarbonate in the presence of a base such as triethylamine in a suitable solvent such as dichloromethane (DCM). Compounds of formula (xxv) may be converted into compounds of formula (xxvi) by treatment with a suitable cyanide source such as zinc cyanide in the presence of a catalyst such as tetrakistriphenylphosphine palladium (0) in a suitable solvent such as dimethylformamide (DMF) at an elevated temperature such as 80° C. Compounds of formula (xxvi) may be converted into compounds of formula (xxvii) by treatment with hydroxylamine hydrochloride as for conversion (xii) to (xiii) in Scheme 1. Compounds of formula (xxvii) may be converted into compounds of formula (xxviii) by treatment with a carboxylic acid chloride of formula (xv) as described for conversion (xiii) to (xvi) in Scheme 1. Compounds of formula (xxviii) may be converted into compounds of formula (xxix) by treatment with a base such as aqueous sodium hydroxide in an alcoholic solvent such as ethanol or methanol. Compounds of formula (xxix) where P is BOC may be converted into certain compounds of formula (I) by treatment with acid, typically trifluoroacetic acid or hydrochloric acid as described in Scheme 1.

One route which may be used to prepare compounds of formula (I) wherein B is

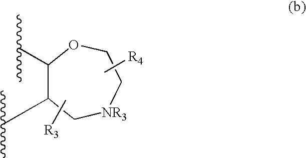

is illustrated in Scheme 3 wherein R₁, R₂ and A are as defined for formula (I), and the side chain R₄ is located in the 5-position of the benzoxazepine ring, R₃ is hydrogen and R₄ is (CH₂)₃CO₂H, R is alkyl (e.g. methyl) and P is protecting group.

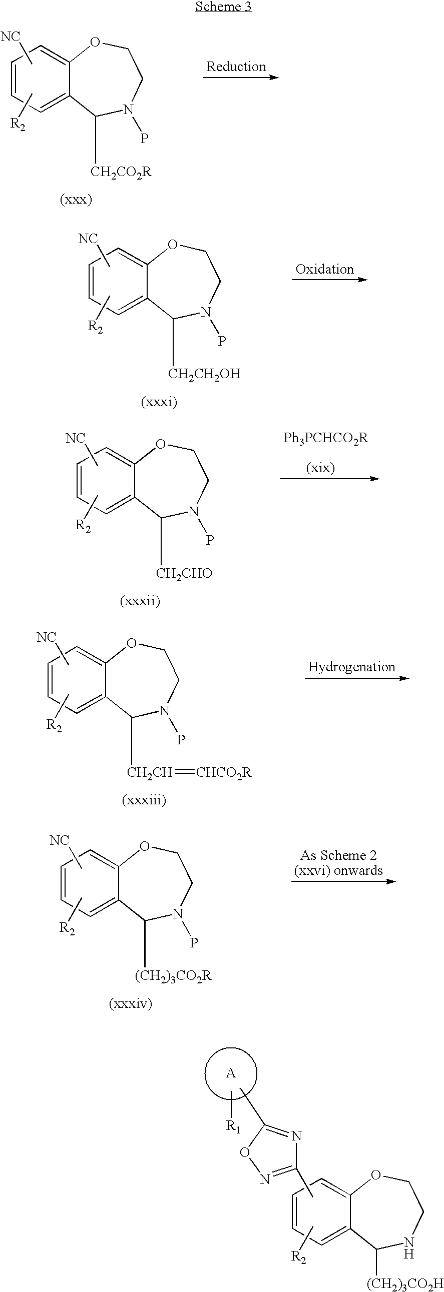

Compounds of formula (xxx) where R₂ is hydrogen and R is alkyl (e.g. ethyl) and P is a Protecting group (e.g. BOC), may be converted into compounds of formula (xxxi) using a suitable reducing agent such as lithium borohydride in a suitable solvent such as methanol. Compounds of formula (xxxi) may be converted into compounds of formula (xxxii) by oxidation using suitable methods such as Swern oxidation. Compounds of formula (xxxii) may be converted into compounds of formula (xxxiii) for example by treatment with a Wittig reagent (xix) such as (carbethoxymethylene)triphenylphosphorane in a suitable solvent such as dichloromethane. Compounds of formula (xxxiii) may be converted into compounds of formula (xxxiv) by hydrogenation using a suitable catalyst such as palladium. Compounds of formula (xxxiv) may be converted into compounds of formula (xxxv) using the methods described for the conversion of compounds of formula (xxvi) to certain compounds of formula (I) in Scheme 2.

One route which may be used to prepare compounds of formula (I) wherein B is

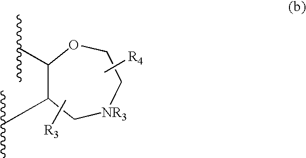

is illustrated in Scheme 4 wherein R₁, R₂ and A are as defined for formula (I) and the side chain R₄ is located in the 5-position of the benzoxazepine ring and is (CH₂)₂CO₂H, R₃ is hydrogen, R is alkyl (e.g. methyl) and P is a protecting group.

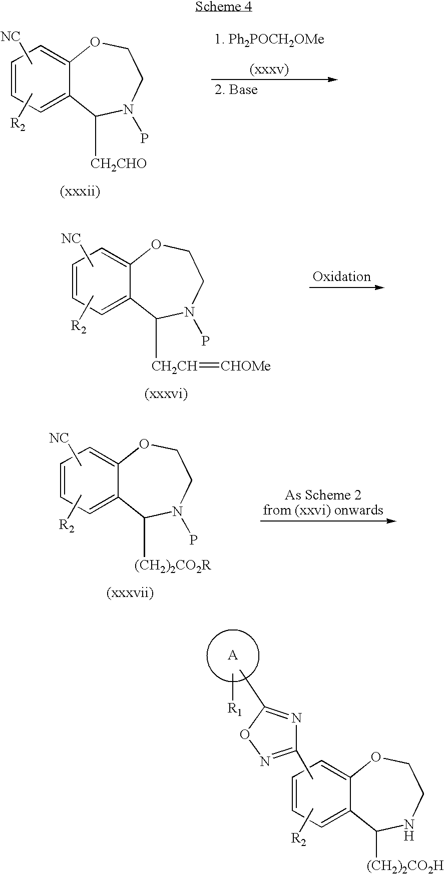

Compounds of formula (xxxii) may be converted into compounds of formula (xxxvi) for example by treatment with a suitable reagent such as a methoxymethyl diphenyl phosphine oxide (xxxv) in the presence of a suitable base such as lithium diisopropylamide (LDA) in a suitable solvent such as THF, followed optionally by the addition of a base such as sodium hydride. Compounds of formula (xxxvi) may be converted into compounds of formula (xxxvii) by oxidation using suitable methods such as pyridinium chlorochromate (PCC) in a suitable solvent such as dichloromethane. Compounds of formula (xxxvii) may be converted into compounds of formula (I) using the methods described for the conversion of compounds of formula (xxvi) to certain compounds of formula (I) in Scheme 2.

One route which may be used to prepare compounds of formula (I) wherein B is

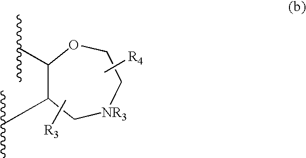

is illustrated in Scheme 5 wherein R₁, R₂ and A are as defined for formula (I) and the side chain R₄ is located in the 3-position of the benzoxazepine ring and is (CH₂)₂CO₂H, R₃ is hydrogen, R is alkyl (e.g. methyl) and P is a protecting group.

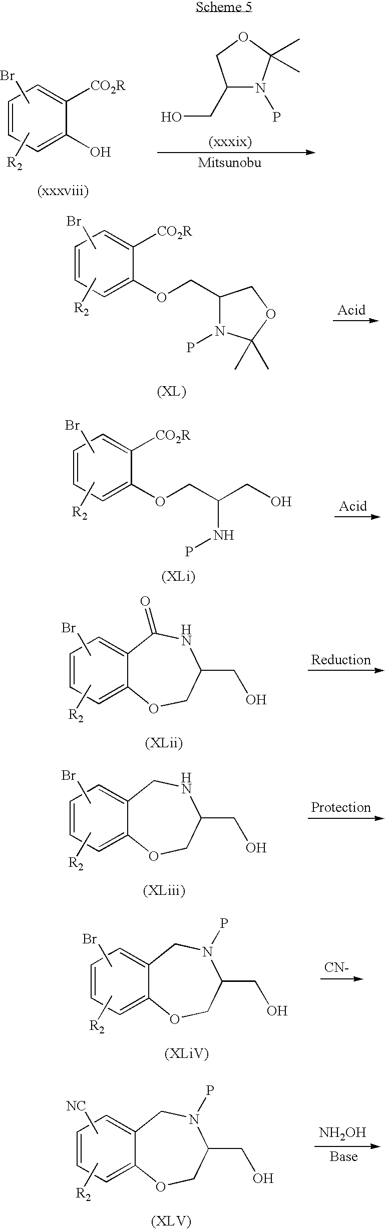

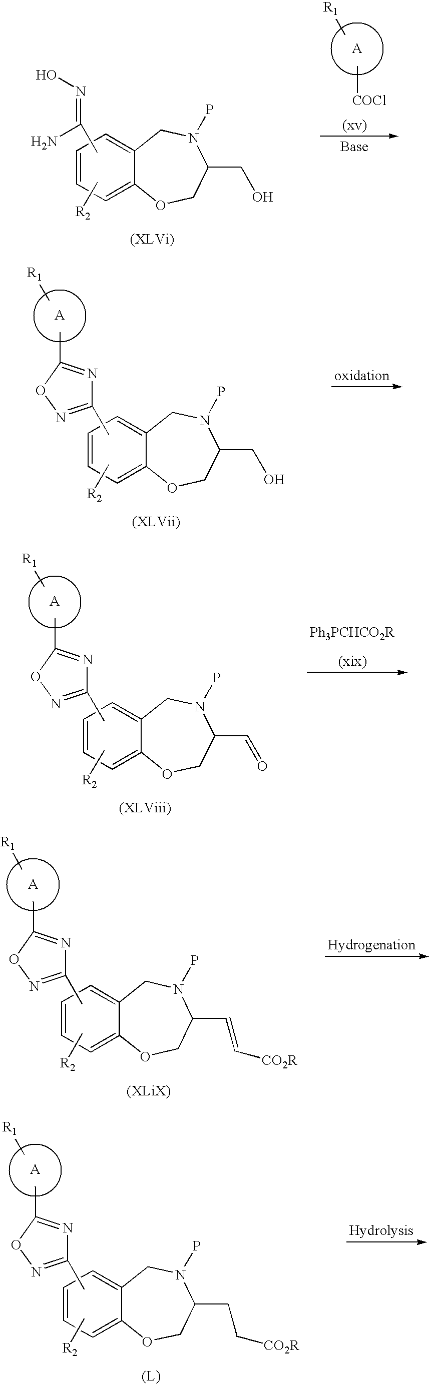

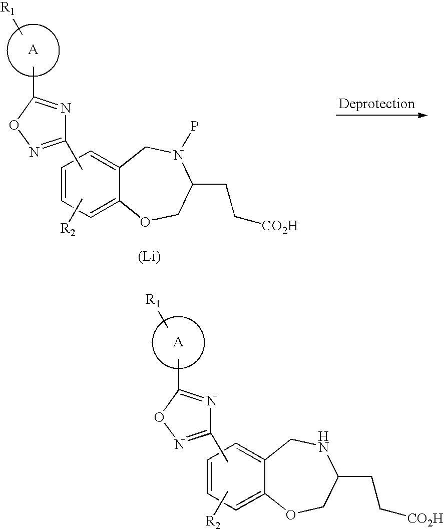

The commercially available compounds (e.g. Aldrich where R=Me) of formula (xxxviii) may be converted into compounds of formula (XL) by treatment with compounds of formula (xxxix) where P is a suitable protecting group such as t-butyloxy carbonyl (BOC) [which may be prepared using, for example the method described in Journal of Organic Chemistry (1987), 52(12), 2361-4], under Mitsunobu conditions using, for example diisopropyl azodicarboxylate (DIAD) and triphenylphosphine in a suitable solvent such as toluene. Compounds of formula (xL) where P represents a suitable protecting group such as t-butyloxy carbonyl (BOC), may be converted into compounds of formula (xLi) by treatment with an acid, typically 4-toluenesulphonic acid in a suitable solvent such as methanol. Compounds of formula (xLi) may be converted into compounds of formula (xLii) by, for example, treatment with an appropriate acid such as trifluoroacetic acid at room temperature, then heated at reflux in the presence of a suitable base such as triethylamine and a suitable solvent such as toluene. Compounds of formula (xLii) may be converted into compounds of formula (xLiii) by treatment with an appropriate reducing agent such as borane-THF complex in a suitable solvent such as THF at a suitable temperature such as at reflux. Compounds of formula (xLiii) may be converted to a protected derivative (xLiv), where P represents a suitable protecting group such as t-butyloxy carbonyl (BOC), for example by treatment with bis(1,1-dimethylethyl) dicarbonate in the presence of a base such as triethylamine in a suitable solvent such as dichloromethane (DCM). Compounds of formula (xLiv) may be converted into compounds of formula (xLv) by treatment with a suitable cyanide source such as zinc cyanide in the presence of a catalyst such as tetrakistriphenylphosphine palladium (0) in a suitable solvent such as dimethylformamide (DMF) at an elevated temperature such as 80° C. Compounds of formula (xLv) may be converted into compounds of formula (xLvi) by treatment with hydroxylamine hydrochloride and an appropriate base, such as sodium bicarbonate, in a solvent such as methanol or ethanol at an elevated temperature such as 60° C.

Compounds of formula (xLvi) may be converted into compounds of formula (xLvii) by treatment with a carboxylic acid chloride of formula (xv) as described in Scheme 1 for conversion of compounds of formula (xiii) to compounds of formula (xvi). Compounds of formula (xLvii) may be converted into compounds of formula (xLviii) by oxidation with a suitable oxidising agent such as Dess-Martin periodinone in a suitable solvent such as dichloromethane. Compounds of formula (xLviii) may be converted into compounds of formula (xLix), for example by treatment with a Wittig reagent (xix) such as (carbethoxymethylene)triphenylphosphorane in a suitable solvent such as dichloromethane. Compounds of formula (xLix) may be converted into compounds of formula (L) by hydrogenation using a suitable catalyst such as palladium in a suitable solvent such as ethanol. Compounds of formula (L) may be converted into compounds of formula (Li) by treatment with a base such as aqueous sodium hydroxide in an alcoholic solvent such as ethanol or methanol. Compounds of formula (Li) where, for example P is BOC may be converted into certain compounds of formula (I) by treatment with acid, typically trifluoroacetic acid or hydrochloric acid as described in Scheme 1.

One route which may be used to prepare compounds of formula (I) wherein B is

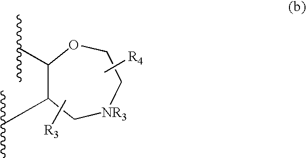

is illustrated in Scheme 6 wherein R₁, R₂ and A are as defined for formula (I) above, and the side chain R₄ is located in the 3-position of the benzoxazepine ring and is represented by CO₂H, R₃ is hydrogen, and P is a protecting group.

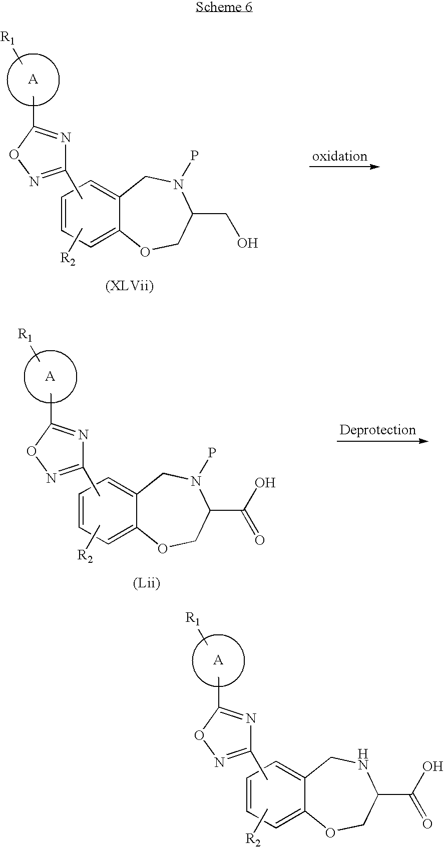

Compounds of formula (xLvii) may be converted into compounds of formula (Lii) by oxidation with a suitable oxidising agent such as chromium trioxide in sulphuric acid in a suitable solvent such as acetone. Compounds of formula (Lii) where, for example P is BOC may be converted into certain compounds of formula (I) by treatment with a suitable acid, typically trifluoroacetic acid or hydrochloric acid.

All publications, including but not limited to patents and patent applications, cited in this specification are herein incorporated by reference as if each individual publication were specifically and individually indicated to be incorporated by reference herein as though fully set forth.

The following Preparations and Examples illustrate the preparation of compounds of the invention.

### ABBREVIATIONS

g—grams
mg—milligrams
ml—millilitres
ul—microlitres
MeCN—acetonitrile
MeOH—methanol
EtOH—ethanol
Et₂O—diethyl ether
EtOAc—ethyl acetate
DCM—dichloromethane
DIAD—diisopropyl azodicarboxylate
DME—1,2-bis(methyloxy)ethane
DMF—N,N-dimethylformamide
DMSO—dimethylsulphoxide
EDAC—N-(3-Dimethylaminopropyl)-N′-ethylcarbodiimide hydrochloride
EDC—N-(3-Dimethylaminopropyl)-N′-ethylcarbodiimide hydrochloride
EDCl—N-(3-Dimethylaminopropyl)-N′-ethylcarbodiimide hydrochloride
HOBT/HOBt—Hydroxybenzotriazole
IPA—isopropylalcohol
NCS—N-chlorosuccinimide
PyBOP—Benzotriazol-1-yl-oxytripyrrolidinophosphonium hexafluorophosphate
THF—tetrahydrofuran
dba—dibenzylidene acetone
RT—room temperature
° C.—degrees Celsius
M—Molar
H—proton
s—singlet
d—doublet
t—triplet
q—quartet
MHz—megahertz
MeOD—deuterated methanol
LCMS—Liquid Chromatography Mass Spectrometry
LC/MS—Liquid Chromatography Mass Spectrometry
MS—mass spectrometry
ES—Electrospray
MH⁺—mass ion+H⁺
MDAP—mass directed automated preparative liquid chromatography.
sat.—saturated

### General Chemistry Section

The methods described below are given for illustrative purposes, intermediates in the preparation of the examples may not necessarily have been prepared from the specific batches described.

### Preparation 1

### Phenylmethyl 3-{3-[(phenylmethyl)oxy]phenyl}propanoate

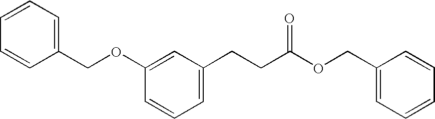

A mixture of 3-(3-hydroxyphenyl)propanoic acid (Fluorochem, 12.46 g, 75 mmol), potassium carbonate (31.1 g, 225 mmol), and benzyl bromide (18.7 ml, 158 mmol) in DMF (120 ml) was stirred at 65° C. for 16 h. The mixture was cooled to room temperature, diluted with ether (200 ml) and water (400 ml) and the aq re-extracted with ether (100 ml). The combined organics were washed with water (3×100 ml), dried (magnesium sulphate) and evaporated to give an almost colourless oil (26.5 g) which was used without purification. ¹H NMR (CDCl₃) δ 2.68 (2H, t), 2.94 (2H, t), 5.01 (2H, s), 5.11 (2H, s), 6.78-6.82 (3H, m), 7.17-7.43 (11H, m).

### Preparation 2

### 3-{3-[(Phenylmethyl)oxy]phenyl}propanoic acid

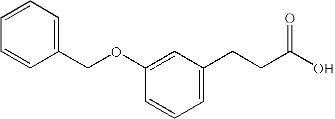

A mixture of phenylmethyl 3-{3-[(phenylmethyl)oxy]phenyl}propanoate (26.5 g, 76 mmol) and 2M aq sodium hydroxide (115 ml, 229 mmol) in ethanol (250 ml) was stirred at 60° C. for 30 min then the ethanol evaporated. The residue was diluted with water (200 ml) and extracted with ether (2×150 ml). The aq phase was acidified with 2M aq HCl and extracted with ether (250 ml and 100 ml) then the combined ether extracts were dried (magnesium sulphate) and evaporated to give a white solid (19.6 g). m/z (ES−) 255 [M−H]⁻.

### Preparation 3

### 3-{3-[(Phenylmethyl)oxy]phenyl}propanamide

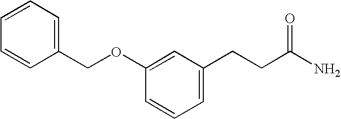

Ethyl chloroformate (8.08 ml, 84 mmol) was added dropwise to a solution of 3-{3-[(phenylmethyl)oxy]phenyl}propanoic acid (19.6 g, 76 mmol) and triethylamine (11.7 ml, 84 mmol) in THF (250 ml) at −10° C. producing a white suspension. The mixture was stirred at −10° C. for 15 min then ammonia (120 ml, 1830 mmol) was added in one portion and allowed to warm to room temperature over 2 h. The THF was evaporated off and the aqueous suspension extracted with DCM (200 ml and 2×50 ml). The combined organics were dried (magnesium sulphate), evaporated, suspended in ether and filtered off to give a white solid (16.2 g). m/z (ES+) 256 [M+H]⁺.

### Preparation 4

### (3-{3-[(Phenylmethyl)oxy]phenyl}propyl)amine

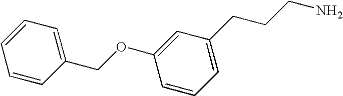

A solution of 3-{3-[(phenylmethyl)oxy]phenyl}propanamide (16.13 g, 63.2 mmol) in THF (350 ml) was added over 15 min to a stirred solution of LiAlH₄ (1M solution in THF, 126 ml, 126 mmol) under argon with cooling in an ice bath. The mixture was then stirred at 65° C. for 2 h, before being cooled to 0° C. and quenched by careful addition of water (4.8 ml), 15% aq sodium hydroxide (4.8 ml) and water (14.4 ml) all with ice cooling. The mixture was filtered, evaporated, re-dissolved in DCM (200 ml), then washed with water (100 ml), dried (magnesium sulphate) and evaporated to give a waxy solid. This was partitioned between ethyl acetate (200 ml) and 2M aq sodium hydroxide (150 ml), then filtered and separated. The organic phase was dried (magnesium sulphate) and evaporated to give a colourless oil (14.1 g). m/z (ES+) 242 [M+H]⁺.

### Preparation 5

### Methyl 4-oxo-4-[(3-{3-[(phenylmethyl)oxy]phenyl}propyl)amino]butanoate

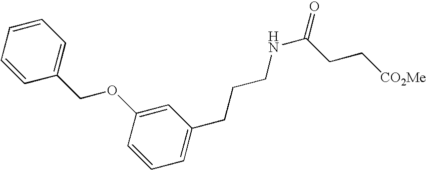

Methyl 4-chloro-4-oxobutanoate (1.58 g, 10.5 mmol) was added dropwise to a stirred solution of (3-{3-[(phenylmethyl)oxy]phenyl}propyl)amine (2.41 g, 9.99 mmol) and triethylamine (1.53 ml, 11.0 mmol) in DCM (30 ml) with ice bath cooling. The mixture was stirred for 30 min then washed with 2M aq hydrochloric acid (30 ml), dried (magnesium sulphate), evaporated and purified by flash chromatography (ethyl acetate/iso-hexane, 1:1) to give a gum which crystallised on scratching (1.91 g). m/z (ES+) 356 [M+H]⁺.

### Preparation 6

### Methyl 3-{7-[(phenylmethyl)oxy]-2,3,4,5-tetrahydro-1H-2-benzazepin-1-yl}propanoate

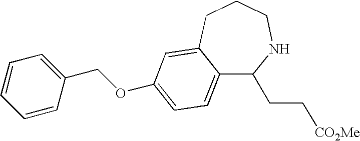

A solution of methyl 4-oxo-4-[(3-{3-[(phenylmethyl)oxy]phenyl}propyl)amino]butanoate (10.4 g, 29.3 mmol) and phosphorus oxychloride (8.95 ml, 96 mmol) in MeCN (250 ml) was stirred at reflux under argon for 2.5 hours then cooled and evaporated. The residue was dissolved in methanol (150 ml), cooled to 0° C. then sodium borohydride (7.65 g, 202 mmol) was added in portions over 25 min and the mixture left to stand for 16 h. Water (600 ml) was added, then the mixture extracted with DCM (200 ml and 2×75 ml). The combined organics were dried (magnesium sulphate), evaporated and purified by flash chromatography (3% methanol in DCM, then 10% methanol in DCM) to give a viscous light brown gum (4.2 g, LCMS: 62% pure). Used in Preparation 7 without further purification. m/z (ES+) 340 [M+H]⁺.

### Preparation 7

### 1,1-Dimethylethyl 1-[3-(methyloxy)-3-oxopropyl]-7-[(phenyl methyl)oxy]-1,3,4,5-tetrahydro-2H-2-benzazepine-2-carboxylate

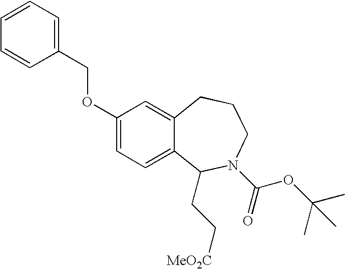

BOC-anhydride (3.16 ml, 13.6 mmol) was added to a solution of methyl 3-{7-[(phenylmethyl)oxy]-2,3,4,5-tetrahydro-1H-2-benzazepin-1-yl}propanoate (4.2 g, 12.4 mmol) and triethylamine (2.07 ml, 14.9 mmol) in DCM (50 ml) and stirred at room temperature for 1 h. The reaction mixture was washed with water (50 ml), dried (magnesium sulphate), evaporated and purified by flash chromatography twice (ethyl acetate/iso-hexane, 1:4) to give a colourless gum (1.81 g). LCMS: 4% of impurity remained. Used in Preparation 8 without further purification. m/z (ES+) 440 [M+H]⁺.

### Preparation 8

### 1,1-Dimethylethyl 7-hydroxy-1-[3-(methyloxy)-3-oxopropyl]-1,3,4,5-tetrahydro-2H-2-benzazepine-2-carboxylate

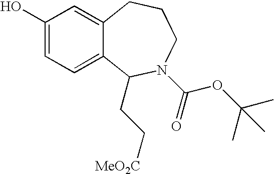

A solution of 1,1-dimethylethyl 1-[3-(methyloxy)-3-oxopropyl]-7-[(phenylmethyl)oxy]-1,3,4,5-tetrahydro-2H-2-benzazepine-2-carboxylate (0.088 g, 0.200 mmol) in methanol (4 ml) was hydrogenated under continuous flow conditions at 50° C. Evaporation gave a colourless gum (55 mg). m/z (ES+) 350 [M+H]⁺.

### Preparation 9

### 1,1-Dimethylethyl 1-[3-(methyloxy)-3-oxopropyl]-7-{[(trifluoromethyl)sulfonyl]oxy}-1,3,4,5-tetrahydro-2H-2-benzazepine-2-carboxylate

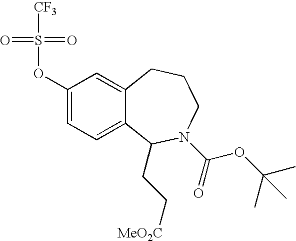

Trifluoromethanesulfonic anhydride (0.041 ml, 0.240 mmol) was added to a solution of 1,1-dimethylethyl 7-hydroxy-1-[3-(methyloxy)-3-oxopropyl]-1,3,4,5-tetrahydro-2H-2-benzazepine-2-carboxylate (0.070 g, 0.200 mmol) in pyridine (2 ml) and stirred at room temperature for 30 min. Ether was added (40 ml), then the mixture washed with 2M aq HCl (20 ml), water (20 ml), dried (magnesium sulphate) and evaporated to give a pale yellow gum (95 mg). m/z (ES+) 482 [M+H]⁺.

### Preparation 10

### 1,1-Dimethylethyl 7-cyano-1-[3-(methyloxy)-3-oxopropyl]-1,3,4,5-tetrahydro-2H-2-benzazepine-2-carboxylate

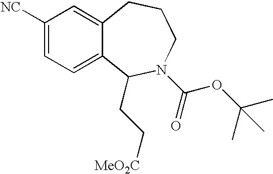

A mixture of 1,1-dimethylethyl 1-[3-(methyloxy)-3-oxopropyl]-7-{[(trifluoromethyl) sulfonyl]oxy}-1,3,4,5-tetrahydro-2H-2-benzazepine-2-carboxylate (1.8 g, 3.74 mmol), zinc cyanide (0.878 g, 7.48 mmol) and tetrakis(triphenylphosphine)palladium (0.864 g, 0.748 mmol) in DMF (15 ml) was stirred at 90° C. under argon for 18 h then cooled to room temperature and diluted with water/ethyl acetate (60 ml each). The organic layer was washed with water (3×20 ml), dried (magnesium sulphate), and purified by flash chromatography (ethyl acetate/iso-hexane, 1:3) to give a colourless gum (608 mg). m/z (ES+) 359 [M+H]⁺.

### Preparation 11

### 1,1-Dimethylethyl 7-[(hydroxyamino)(imino)methyl]-1-[3-(methyloxy)-3-oxopropyl]-1,3,4,5-tetrahydro-2H-2-benzazepine-2-carboxylate

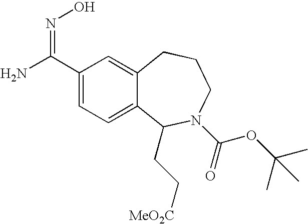

A mixture of 1,1-dimethylethyl 7-cyano-1-[3-(methyloxy)-3-oxopropyl]-1,3,4,5-tetrahydro-2H-2-benzazepine-2-carboxylate (0.072 g, 0.201 mmol), hydroxylamine hydrochloride (0.028 g, 0.402 mmol), and sodium bicarbonate (0.067 g, 0.804 mmol) in ethanol (4 ml) was stirred at 60° C. for 3 h then left at room temperature for 64 h. Ethyl acetate (40 ml) was added and the mixture washed with water (50 ml), dried (magnesium sulphate) and evaporated to give a colourless gum (80 mg, LCMS: 81% pure). m/z (ES+) 392 [M+H]⁺.

### Preparation 12

### 1,1-Dimethylethyl 7-(5-{3-chloro-4-[(1-methylethyl)oxy]phenyl}-1,2,4-oxadiazol-3-yl)-1-[3-(methyloxy)-3-oxopropyl]-1,3,4,5-tetrahydro-2H-2-benzazepine-2-carboxylate

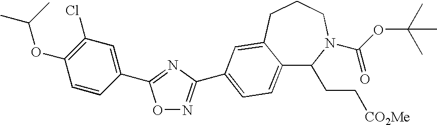

A mixture of 1,1-dimethylethyl 7-[(hydroxyamino)(imino)methyl]-1-[3-(methyloxy)-3-oxopropyl]-1,3,4,5-tetrahydro-2H-2-benzazepine-2-carboxylate (0.57 g, 1.46 mmol), 3-chloro-4-[(1-methylethyl)oxy]benzoic acid (Paragos Product, 0.344 g, 1.60 mmol), EDC (0.307 g, 1.60 mmol) and HOBt (0.245 g, 1.60 mmol) in DMF (8 ml) was stirred at room temperature for 20 min, then heated at 120° C. for 3 h. The reaction was cooled to room temperature and diluted with ethyl acetate/water (80 ml each). The organic phase was washed with saturated aqueous sodium bicarbonate and water (3×20 ml), then dried (magnesium sulphate), evaporated and purified by flash chromatography (ethyl acetate/iso-hexane, 1:3) to give a white foam (515 mg). m/z (ES+) 570 [M+H]⁺.

### Preparation 13

### 3-(7-(5-{3-Chloro-4-[(1-methylethyl)oxy]phenyl}-1,2,4-oxadiazol-3-yl)-2-{[(1,1-dimethylethyl)oxy]carbonyl}-2,3,4,5-tetrahydro-1H-2-benzazepin-1-yl)propanoic acid

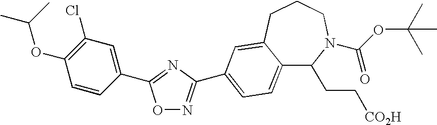

2M aq sodium hydroxide (0.5 ml, 1.0 mmol) was added to a solution of 1,1-dimethylethyl 7-(5-{3-chloro-4-[(1-methylethyl)oxy]phenyl}-1,2,4-oxadiazol-3-yl)-1-[3-(methyloxy)-3-oxopropyl]-1,3,4,5-tetrahydro-2H-2-benzazepine-2-carboxylate (0.090 g, 0.16 mmol) in ethanol (4 ml) and stirred at room temperature for 17 h. The mixture was evaporated and the residue partitioned between water and ether, then acidified with 2M aq HCl. The organic phase was dried (magnesium sulphate) and evaporated to give a white foam (86 mg). m/z (ES+) 556 [M+H]⁺.

### Preparation 14

### Methyl 3-[7-(5-{3-chloro-4-[(1-methylethyl)oxy]phenyl}-1,2,4-oxadiazol-3-yl)-2,3,4,5-tetrahydro-1H-2-benzazepin-1-yl]propanoate

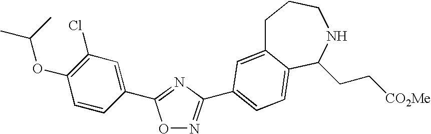

Trifluoroacetic acid (1 ml, 12.98 mmol) was added to a solution of 1,1-dimethylethyl 7-(5-{3-chloro-4-[(1-methylethyl)oxy]phenyl}-1,2,4-oxadiazol-3-yl)-1-[3-(methyloxy)-3-oxopropyl]-1,3,4,5-tetrahydro-2H-2-benzazepine-2-carboxylate (0.10 g, 0.175 mmol) in DCM (1 ml) and stirred at room temperature for 30 min. The solution was evaporated, re-dissolved in ether (30 ml), washed with 1M aq potassium carbonate, dried (magnesium sulphate) and evaporated to give a colourless gum (71 mg). m/z (ES+) 470 [M+H]⁺.

### Preparation 15

### 1,1-Dimethylethyl 4-[({4-bromo-2-[(methyloxy)carbonyl]phenyl}oxy)methyl]-2,2-dimethyl-1,3-oxazolidine-3-carboxylate

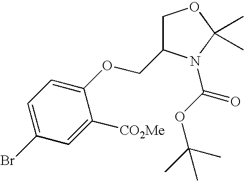

DIAD (6.40 ml, 32.9 mmol) was added to a solution of 1,1-dimethylethyl 4-(hydroxymethyl)-2,2-dimethyl-1,3-oxazolidine-3-carboxylate (6.92 g, 29.9 mmol), methyl 5-bromo-2-hydroxybenzoate (6.91 g, 29.9 mmol) and triphenylphosphine (8.63 g, 32.9 mmol) in toluene (70 ml) then the mixture stirred at 80° C. for 18 h. The solution was cooled, evaporated and purified by flash chromatography (ethyl acetate/iso-hexane, 1:9) to give a colourless oil (7.56 g). m/z (ES+) 344 and 346 (1:1, [M+H-100]⁺).

### Preparation 16

### Methyl 5-bromo-2-{[2-({[(1,1-dimethylethyl)oxy]carbonyl}amino)-3-hydroxypropyl]oxy}benzoate

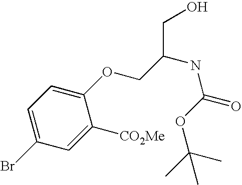

To a solution of 1,1-dimethylethyl 4-[({4-bromo-2-[(methyloxy)carbonyl]phenyl}oxy)methyl]-2,2-dimethyl-1,3-oxazolidine-3-carboxylate (7.54 g, 17.0 mmol) in methanol (100 ml) was added p-toluenesulfonic acid (0.10 g, 0.53 mmol) and the mixture stirred at room temperature for 16 h. The solution was evaporated to dryness and purified by flash chromatography (ethyl acetate/iso-hexane, 2:3) to give a colourless viscous oil (5.55 g). m/z (ES+) 404 and 406 (1:1, [M+H]⁺).

### Preparation 17

### 7-Bromo-3-(hydroxymethyl)-3,4-dihydro-1,4-benzoxazepin-5(2H)-one

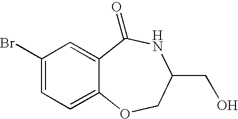

Methyl 5-bromo-2-{[2-({[(1,1-dimethylethyl)oxy]carbonyl}amino)-3-hydroxypropyl]oxy}benzoate (5.52 g, 13.7 mmol) was dissolved in DCM (15 ml) and TFA (15 ml, 195 mmol) and stirred at room temperature for 1 h. The solution was evaporated, then the residue re-dissolved in toluene (60 ml) and triethylamine (8 ml, 57.4 mmol) before being heated at reflux for 2 h. The mixture was evaporated to dryness, re-dissolved in ethyl acetate (100 ml), washed with water (20 ml), dried (magnesium sulphate) and evaporated. The residue was dissolved in 2M ammonia in methanol (50 ml) and stirred at room temperature for 5 days. Evaporation and purification by flash chromatography (3.5% methanol in DCM) gave a white solid (2.71 g). m/z (ES+) 272 and 274 (1:1, [M+H]⁺).

### Preparation 18

### (7-Bromo-2,3,4,5-tetrahydro-1,4-benzoxazepin-3-yl)methanol

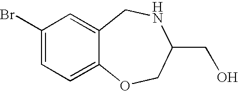

1M Borane-THF complex in THF (26.0 ml, 26.0 mmol) was added to a suspension of 7-bromo-3-(hydroxymethyl)-3,4-dihydro-1,4-benzoxazepin-5(2H)-one (2.36 g, 8.67 mmol) in THF (5 ml) and the mixture heated at reflux for 5 h, then cooled to room temperature. A 5M aq HCl solution (50 ml) was added and the mixture heated at reflux for 1 h, then cooled to room temperature. Evaporation and azeotropic distillation of residual solvent with ethanol (100 ml) left a residue that was redissolved in ethanol (100 ml) and triethylamine (20 ml), then concentrated again. Purification by flash chromatography (5% methanol in DCM) gave a crude product that was partitioned between ethyl acetate (300 ml) and water (100 ml). This mixture was basified with 2M aq sodium hydroxide, then the organics dried (magnesium sulphate) and evaporated to give a white solid (1.93 g). m/z (ES+) 258 and 260 (1:1, [M+H]⁺).

### Preparation 19

### 1,1-Dimethylethyl 7-bromo-3-(hydroxymethyl)-2,3-dihydro-1,4-benzoxazepine-4(5H)-carboxylate

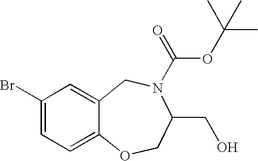

A mixture of (7-bromo-2,3,4,5-tetrahydro-1,4-benzoxazepin-3-yl)methanol (1.93 g, 7.48 mmol) and BOC-anhydride (2.08 ml, 8.97 mmol) in THF (30 ml) and saturated aq sodium bicarbonate (30 ml) was stirred at room temperature for 2 h. The mixture was diluted with ethyl acetate (100 ml), washed with water (50 ml), dried (magnesium sulphate), evaporated and purified by flash chromatography (ethyl acetate/iso-hexane, 2:3) to give white solid (2.36 g). m/z (ES+) 258 and 260 (1:1, [M+H−100]⁺).

### Preparation 20

### 1,1-Dimethylethyl 7-cyano-3-(hydroxymethyl)-2,3-dihydro-1,4-benzoxazepine-4(5H)-carboxylate

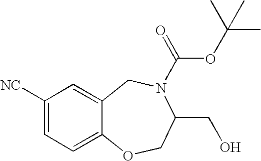

A mixture of 1,1-dimethylethyl 7-bromo-3-(hydroxymethyl)-2,3-dihydro-1,4-benzoxazepine-4(5H)-carboxylate (2.05 g, 5.72 mmol), zinc cyanide (1.34 g, 11.5 mmol) and tetrakis(triphenylphosphine)palladium (0.992 g, 0.858 mmol) in DMF (20 ml) was stirred at 80° C. under argon for 3 h. The mixture was cooled, diluted with ethyl acetate/water (150 ml each) and the layers separated. The organic phase was washed with water (3×30 ml), dried (magnesium sulphate), evaporated and purified by flash chromatography (ethyl acetate/iso-hexane, 1:1) to give a white solid (1.43 g). m/z (ES+) 205 [M+H−100]⁺.

### Preparation 21

### 1,1-Dimethylethyl 7-[(hydroxyamino)(imino)methyl]-3-(hydroxymethyl)-2,3-dihydro-1,4-benzoxazepine-4(5H)-carboxylate

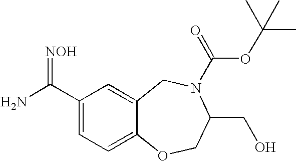

A mixture of 1,1-dimethylethyl 7-cyano-3-(hydroxymethyl)-2,3-dihydro-1,4-benzox-azepine-4(5H)-carboxylate (1.43 g, 4.70 mmol), hydroxylamine hydrochloride (0.653 g, 9.40 mmol) and sodium bicarbonate (1.974 g, 23.5 mmol) in ethanol (20 ml) was stirred at 50° C. for 15 h then cooled and diluted with ethyl acetate/water (100 ml each). The aqueous was re-extracted with ethyl acetate (50 ml), then the combined organics dried (magnesium sulphate) and evaporated to give a white foam (1.60 g). m/z (ES+) 338 [M+H]⁺.

### Preparation 22

### 1,1-Dimethylethyl 7-(5-{3-chloro-4-[(1-methylethyl)oxy]phenyl}-1,2,4-oxadiazol-3-yl)-3-(hydroxymethyl)-2,3-dihydro-1,4-benzoxazepine-4(5H)-carboxylate

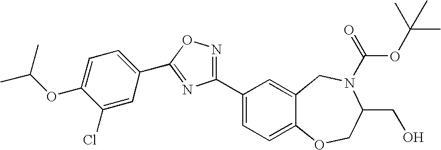

A mixture of 1,1-dimethylethyl 7-[(hydroxyamino)(imino)methyl]-3-(hydroxymethyl)-2,3-dihydro-1,4-benzoxazepine-4(5H)-carboxylate (1.59 g, 4.71 mmol), 3-chloro-4-[(1-methylethyl)oxy]benzoic acid (Paragos Product, 1.012 g, 4.71 mmol), HOBt (0.794 g, 5.18 mmol) and EDC (0.994 g, 5.18 mmol) in DMF (20 ml) was stirred at room temperature for 30 min then heated at 100° C. for 5 h. The mixture was cooled, diluted with ethyl acetate (150 ml), then washed with water (100 ml), saturated aq sodium bicarbonate solution and water (3×50 ml). Evaporation then purification by flash chromatography (ethyl acetate/iso-hexane, 45:55) gave a white solid (673 mg). m/z (ES+) 516 [M+H]⁺.

### Preparation 23

### 7-(5-{3-Chloro-4-[(1-methylethyl)oxy]phenyl}-1,2,4-oxadiazol-3-yl)-4-{[(1,1-dimethylethyl)oxy]carbonyl}-2,3,4,5-tetrahydro-1,4-benzoxazepine-3-carboxylic acid

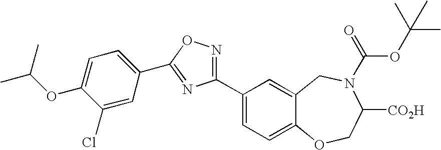

Jones reagent [prepared from chromium trioxide (0.213 g, 2.13 mmol) and sulphuric acid (184 μl, 3.45 mmol) made up to 0.8 ml with water], was added to an ice cooled solution of 1,1-dimethylethyl 7-(5-{3-chloro-4-[(1-methylethyl)oxy]phenyl}-1,2,4-oxadiazol-3-yl)-3-(hydroxymethyl)-2,3-dihydro-1,4-benzoxazepine-4(5H)-carboxylate (0.103 g, 0.2 mmol) in acetone (4 ml) and the resulting mixture stirred in ice for 2 h. IPA (0.5 ml) was added, followed by water (20 ml) and ethyl acetate (40 ml). The organic phase was dried (magnesium sulphate), evaporated and purified by flash chromatography (ethyl acetate/iso-hexane, 1:1 then 1% acetic acid in ethyl acetate/iso-hexane, 1:1) gave a white solid (72 mg). m/z (ES+) 530 [M+H]⁺.

### Preparation 24

### 1,1-Dimethylethyl 7-(5-{3-chloro-4-[(1-methylethyl)oxy]phenyl}-1,2,4-oxadiazol-3-yl)-3-formyl-2,3-dihydro-1,4-benzoxazepine-4(5H)-carboxylate

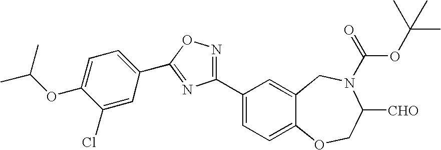

Dess-Martin periodinane (0.127 g, 0.300 mmol) was added to a solution of 1,1-dimethylethyl 7-(5-{3-chloro-4-[(1-methylethyl)oxy]phenyl}-1,2,4-oxadiazol-3-yl)-3-(hydroxymethyl)-2,3-dihydro-1,4-benzoxazepine-4(5H)-carboxylate (0.155 g, 0.30 mmol) in DCM (5 ml) under argon and the resulting mixture stirred at room temperature for 1 h. DCM (30 ml) was added, then the mixture washed with a solution of sodium thiosulphate (3 g) in saturated aq sodium bicarbonate (20 ml). The organic phase was dried (magnesium sulphate), evaporated and purified by flash chromatography (ethyl acetate/iso-hexane, 2:3) to give a gum which was triturated with ether to leave a white solid (136 mg). m/z (ES+) 513 [M⁺].

### Preparation 25

### 1,1-Dimethylethyl 7-(5-{3-chloro-4-[(1-methylethyl)oxy]phenyl}-1,2,4-oxadiazol-3-yl)-3-[(1E)-3-(ethyloxy)-3-oxo-1-propen-1-yl]-2,3-dihydro-1,4-benzoxazepine-4(5H)-carboxylate

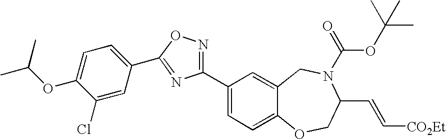

A mixture of 1,1-dimethylethyl 7-(5-{3-chloro-4-[(1-methylethyl)oxy]phenyl}-1,2,4-oxadiazol-3-yl)-3-formyl-2,3-dihydro-1,4-benzoxazepine-4(5H)-carboxylate (0.136 g, 0.265 mmol) and ethyl (triphenyl-15-phosphanylidene)acetate (0.101 g, 0.291 mmol) in THF (4 ml) was stirred at 40° C. under argon for 1 h. Evaporation and purification by flash chromatography (ethyl acetate/iso-hexane, 1:4) gave a colourless gum (109 mg). m/z (ES+) 584 [M+H]⁺.

### Preparation 26

### 1,1-Dimethylethyl 7-(5-{3-chloro-4-[(1-methylethyl)oxy]phenyl}-1,2,4-oxadiazol-3-yl)-3-[3-(ethyloxy)-3-oxopropyl]-2,3-dihydro-1,4-benzoxazepine-4(5H)-carboxylate

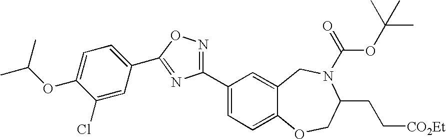

A solution of 1,1-dimethylethyl 7-(5-{3-chloro-4-[(1-methylethyl)oxy]phenyl}-1,2,4-oxadiazol-3-yl)-3-[(1E)-3-(ethyloxy)-3-oxo-1-propen-1-yl]-2,3-dihydro-1,4-benzox-azepine-4(5H)-carboxylate (0.109 g, 0.187 mmol) in ethanol (20 ml) was hydrogenated with 10% Pd/C (0.030 g, 0.282 mmol, 50% water) for 45 min then filtered, evaporated and purified by flash chromatography (ethyl acetate/iso-hexane, 1:4) to give a colourless gum (59 mg). m/z (ES+) 586 [M+H]⁺.

### Preparation 27

### 3-(7-(5-{3-Chloro-4-[(1-methylethyl)oxy]phenyl}-1,2,4-oxadiazol-3-yl)-4-{[(1,1-dimethylethyl)oxy]carbonyl}-2,3,4,5-tetrahydro-1,4-benzoxazepin-3-yl)propanoic acid

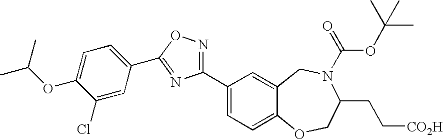

A 2M aqueous sodium hydroxide solution (1.0 ml, 2.0 mmol) was added to a solution of 1,1-dimethylethyl 7-(5-{3-chloro-4-[(1-methylethyl)oxy]phenyl}-1,2,4-oxadiazol-3-yl)-3-[3-(ethyloxy)-3-oxopropyl]-2,3-dihydro-1,4-benzoxazepine-4(5H)-carboxylate (0.059 g, 0.101 mmol) in ethanol (4 ml) and stirred at room temperature for 2 h. The mixture was evaporated, then partitioned between water and ethyl acetate (25 ml of each) and acidified with 2M aq HCl. The organic phase was dried (magnesium sulphate) and evaporated to give a colourless gum (55 mg). m/z (ES−) 556 [M−H]⁻.

### Preparation 28

### Ethyl (2E)-3-(3-bromo-2-hydroxyphenyl)-2-propenoate

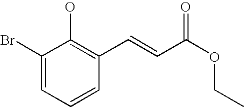

To a solution of 3-bromo-2-hydroxybenzaldehyde (WO9606822; 1.005 g, 5.0 mmol) in dichloromethane (20 ml) at room temperature was added (carbethoxymethylene)triphenylphosphorane (Aldrich; 2.09 g, 6.0 mmol). The reaction was stirred at room temperature for 3 hours, then the solvent was evaporated. Purification of the residue by chromatography, eluting with 20% ethyl acetate in cyclohexane gave the title compound as a white solid (1.2 g). MS (ES) C₁₁H₁₁BrO₃ requires 270, 272; found 271, 273 [M+H]⁺.

### Preparation 29

### Ethyl (2E)-3-(3-bromo-2-{[2-({[(1,1-dimethylethyl)oxy]carbonyl}amino)ethyl]oxy}phenyl)-2-propenoate

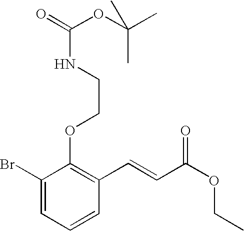

To a solution of ethyl (2E)-3-(3-bromo-2-hydroxyphenyl)-2-propenoate (Preparation 28) (870 mg, 3.21 mmol), 1,1-dimethylethyl (2-hydroxyethyl)carbamate (Aldrich; 517 mg, 3.21 mmol) and triphenylphosphine (926 mg, 3.53 mmol) in tetrahydrofuran (20 ml) at 0° C. was added diisopropyl azodicarboxylate (714 mg, 3.53 mmol). The reaction mixture was stirred at room temperature for 2 hours. The solvent was evaporated and the residue dissolved in ethyl acetate then washed twice with brine. The organic phase was dried and evaporated. Purification by chromatography eluting with 20% ethyl acetate in cyclohexane gave the product as a colourless oil (1.23 g). MS (ES) C₁₈H₂₄BrNO₅ requires 413,415; found 414, 416 [M+H]⁺.

### Preparation 30

### Ethyl (2E)-3-{2-[(2-aminoethyl)oxy]-3-bromophenyl}-2-propenoate trifluoroacetic acid salt

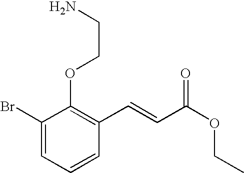

To a solution of ethyl (2E)-3-(3-bromo-2-{[2-({[(1,1-dimethylethyl)oxy]carbonyl}amino)ethyl]oxy}phenyl)-2-propenoate (Preparation 29) (0.207 g, 0.5 mmol) in dichloromethane (4 ml) in an ice bath was slowly added trifluoroacetic acid (1 ml). After 1 hour the reaction mixture was concentrated to dryness, and the residue azeotroped with diethyl ether. The residue was dried under vacuum to give the crude product as as an oil (0.214 g). MS (ES) C₁₃H₁₆BrNO₃ requires 313, 315; found 314, 316 [M+H]⁺.

### Preparation 31

### Ethyl (9-bromo-2,3,4,5-tetrahydro-1,4-benzoxazepin-5-yl)acetate

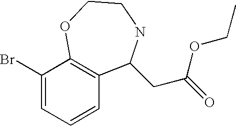

To a solution of ethyl (2E)-3-{2-[(2-aminoethyl)oxy]-3-bromophenyl}-2-propenoate (Preparation 30) (1.4 g, 4.46 mmol) in tetrahydrofuran (40 ml) at room temperature under nitrogen was added 1,8-diazabicyclo[5.4.0]undec-7-ene (0.678 g, 4.46 mmol) and the reaction mixture was stirred at room temperature overnight. The solvent was evaporated and the residue purified by chromatography, eluting with 0-2% methanol in dichloromethane to give the title compound as a colourless oil (1.31 g).

MS (ES) C₁₃H₁₆BrNO₃ requires 313,315; found 314,316 [M+H]⁺.

### Preparation 32

### 1,1-Dimethylethyl 9-bromo-5-[2-(ethyloxy)-2-oxoethyl]-2,3-dihydro-1,4-benzoxazepine-4(5H)-carboxylate

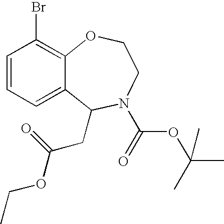

To a solution of ethyl (9-bromo-2,3,4,5-tetrahydro-1,4-benzoxazepin-5-yl)acetate (Preparation 31) (12.3 g, 39.2 mmol) in tetrahydrofuran (200 ml) at 0° C. was added triethylamine (4.77 g, 47.1 mmol) then di-tert-butyl dicarbonate (9.42 g, 43.2 mmol). After 2 min, the ice bath was removed and the reaction mixture left standing at room temperature over the weekend. The solvent was evaporated and the residue purified by chromatography, eluting with 3-15% ethyl acetate in cyclohexane gave the product as a colourless oil (15.7 g). MS (ES) C₁₈H₂₄BrNO₅ requires 413, 415 found; 414, 416 [M+H]⁺.

### Preparation 33

### 1,1-Dimethylethyl 9-cyano-5-[2-(ethyloxy)-2-oxoethyl]-2,3-dihydro-1,4-benzoxazepine-4(5H)-carboxylate

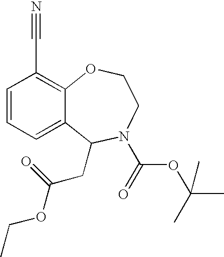

A solution of 1,1-dimethylethyl 9-bromo-5-[2-(ethyloxy)-2-oxoethyl]-2,3-dihydro-1,4-benzoxazepine-4(5H)-carboxylate (Preparation 32) (1.02 g, 2.47 mmol) in N,N-dimethylformamide (DMF) (7 ml) was degassed under vacuum for 15 minutes, then zinc cyanide (348 mg, 2.98 mmol) and tetrakis(triphenylphosphine)palladium(0) (285 mg, 0.247 mmol) were added and the resulting yellow mixture was stirred under nitrogen at 100° C. for 5 hours. The cooled mixture was filtered, and washed with DMF. The filtrate was evaporated and the residue partitioned between ethyl acetate and brine. The combined organic extracts were washed with brine and the organic phase was dried and evaporated. Purification by flash chromatography, eluting with 20% ethyl acetate in cyclohexane gave the title compound as a colourless solid (760 mg). MS (ES) C₁₉H₂₄N₂O₅ requires 360; found 361 [M+H]⁺.

### Preparation 34

### 1,1-Dimethylethyl 5-[2-(ethyloxy)-2-oxoethyl]-9-[-(hydroxyamino)(imino)methyl]-2,3-dihydro-1,4-benzoxazepine-4(5H)-carboxylate

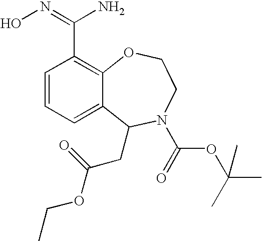

A mixture of 1,1-dimethylethyl 9-cyano-5-[2-(ethyloxy)-2-oxoethyl]-2,3-dihydro-1,4-benzoxazepine-4(5H)-carboxylate (Preparation 33) (1.08 g, 3.0 mmol), hydroxylamine hydrochloride (521 mg, 7.5 mmol), and sodium bicarbonate (630 mg, 7.5 mmol) in ethanol (15 ml) was refluxed for 5 hours. The reaction mixture was cooled to room temperature, filtered through ‘celite’ and the solvent evaporated. The residue was purified by chromatography, eluting with 0-5% methanol in dichloromethane to give the title compound as a colourless foam (0.968 g). MS (ES) C₁₉H₂₇N₃O₆ requires 393; found 394 [M+H]⁺.

### Preparation 35

### 1,1-Dimethylethyl 9-(5-{3-chloro-4-[(1-methylethyl)oxy]phenyl}-1,2,4-oxadiazol-3-yl)-5-[2-(ethyloxy)-2-oxoethyl]-2,3-dihydro-1,4-benzoxazepine-4(5H)-carboxylate

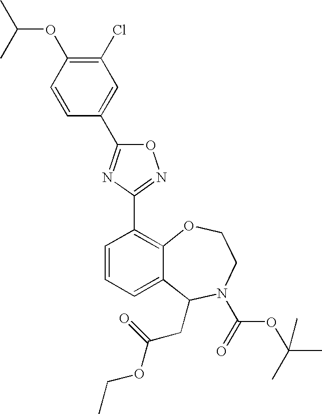

To a solution of 1,1-dimethylethyl 5-[2-(ethyloxy)-2-oxoethyl]-9-[(hydroxyamino)(imino)methyl]-2,3-dihydro-1,4-benzoxazepine-4(5H)-carboxylate (Preparation 34) (826 mg, 2.09 mmol) and triethylamine (255 mg, 2.52 mmol) in N,N-dimethylformamide (21 ml) was added 3-chloro-4-[(1-methylethyl)oxy]benzoyl chloride (Preparation 41) 401 mg, 2.09 mmol) under nitrogen. The mixture was stirred at room temperature for 10 min, then at 120 for 20 min. After 20 min, more triethylamine (51 mg. 0.504 mmol) and 3-chloro-4-[(1-methylethyl)oxy]benzoyl chloride (80.2 mg, 0.418 mmol) were added and the mixture stirred at 120° C. for 6 hours. The mixture was cooled to room temperature and the solvent evaporated. The residue was dissolved in ethyl acetate and the solution washed with 2N sodium hydroxide, then brine, dried (Na₂SO₄) and evaporated. Purification of the residue by chromatography, eluting with 20% ethyl acetate in cyclohexane gave the title compound as a foam (740 mg). MS (ES) C₂₉H₃₄³⁵ClN₃O₇ requires 571; found 572 [M+H]⁺.

### Preparation 36

### (9-(5-{3-Chloro-4-[(1-methylethyl)oxy]phenyl}-1,2,4-oxadiazol-3-yl)-4-{[(1,1-dimethylethyl)oxy]carbonyl}-2,3,4,5-tetrahydro-1,4-benzoxazepin-5-yl)acetic acid sodium salt

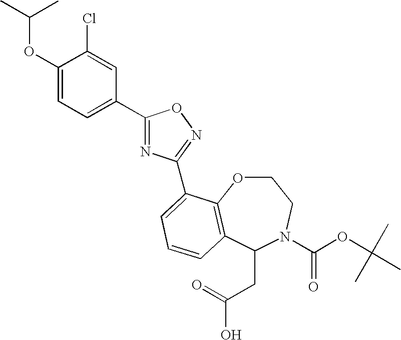

A mixture of 1,1-dimethylethyl 9-(5-{3-chloro-4-[(1-methylethyl)oxy]phenyl}-1,2,4-oxadiazol-3-yl)-5-[2-(ethyloxy)-2-oxoethyl]-2,3-dihydro-1,4-benzoxazepine-4(5H)-carboxylate (Preparation 35) (120 mg, 0.21 mmol), ethanol (1 ml) and 2M sodium hydroxide (2 ml) was stirred at 60° C. for 1 hour. The reaction mixture was cooled and the solvent evaporated. The residue was suspended in water (2 ml) and extracted with ethyl acetate (2×2 ml). The combined extracts were dried (Na₂SO₄) and evaporated. Trituration of the residue with diethyl ether gave the title compound as a colourless solid (90 mg).

MS (ES) C₂₇H₃₀ClN₃O₇ requires 543 found 544 [M+H]⁺.

### Preparation 37

### 1,1-Dimethylethyl 9-(5-{3-cyano-4-[(1-methylethyl)oxy]phenyl}-1,2,4-oxadiazol-3-yl)-5-[2-(ethyloxy)-2-oxoethyl]-2,3-dihydro-1,4-benzoxazepine-4(5H)-carboxylate

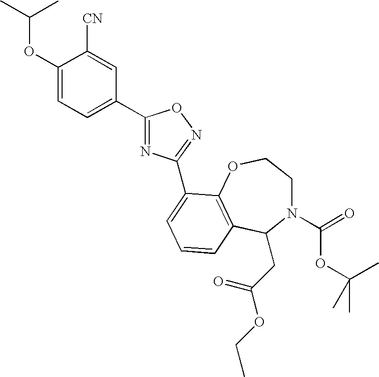

i) Oxalyl chloride (152 mg, 105 μl, 1.2 mmol) was added to a stirred solution of 3-cyano-4-[(1-methylethyl)oxy]benzoic acid (WO2001002355; 205 mg, 1 mmol) in dry dichloromethane (5 ml) followed by N,N-dimethylformamide (1 drop, catalyst) and the reaction mixture was stirred at room temperature for 1 hour. The solvent was then evaporated and the residue dried under vacuum for 30 minutes.

ii) A solution of the crude acid chloride (step i) (1 mmol) in acetonitrile (5 ml) was added dropwise to a stirred solution of 1,1-dimethylethyl 5-[2-(ethyloxy)-2-oxoethyl]-9-[(hydroxyamino)(imino)methyl]-2,3-dihydro-1,4-benzoxazepine-4(5H)-carboxylate (Preparation 34) (393 mg, 1 mmol) and triethylamine (121 mg, 167 μl, 1.2 mmol) in acetonitrile (5 ml) and the mixture stirred at room temperature for 1 hour, then heated under reflux for 72 hours. The reaction mixture was cooled and the solvent evaporated. The residue was purified by chromatography, eluting with 15-25% ethyl acetate in cyclohexane to give the title compound as a colourless oil (180 mg).

MS (ES) C₃₀H₃₄N₄O₇ requires 562 found 563 [M+H]⁺.

### Preparation 38

### (9-(5-{3-Cyano-4-[(1-methylethyl)oxy]phenyl}-1,2,4-oxadiazol-3-yl)-4-{[(1,1-dimethylethyl)oxy]carbonyl}-2,3,4,5-tetrahydro-1,4-benzoxazepin-5-yl)acetic acid

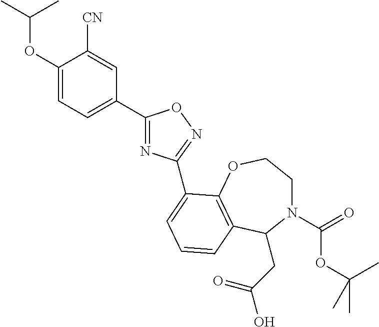

A mixture of 1,1-dimethylethyl 9-(5-{3-cyano-4-[(1-methylethyl)oxy]phenyl}-1,2,4-oxadiazol-3-yl)-5-[2-(ethyloxy)-2-oxoethyl]-2,3-dihydro-1,4-benzoxazepine-4(5H)-carboxylate (Preparation 37) (170 mg, 0.30 mmol), 2M sodium hydroxide (2 ml) and ethanol (4 ml) was stirred at 60° C. for 1 hour. The reaction mixture was cooled to room temperature and the ethanol evaporated. The residue was diluted with water (5 ml) and acidified with glacial acetic acid. The mixture was extracted with ethyl acetate (3×5 ml). The combined extracts were dried (Na₂SO₄) and evaporated. The residue was dried under high vacuum to give the title compound as a colourless foam (150 mg). MS (ES) C₂₈H₃₀N₄O₇ requires 534 found 535 [M+H]⁺.

### Preparation 39

### 1,1-Dimethylethyl 9-(5-{5-chloro-6-[(1-methylethyl)oxy]-3-pyridinyl}-1,2,4-oxadiazol-3-yl)-5-[2-(ethyloxy)-2-oxoethyl]-2,3-dihydro-1,4-benzoxazepine-4(5H)-carboxylate

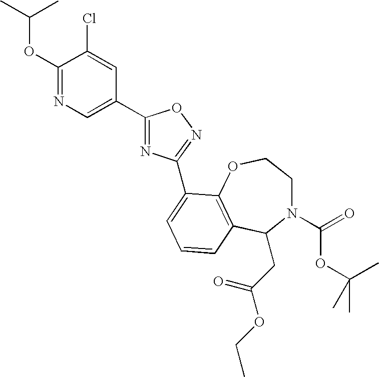

i) Oxalyl chloride (152 mg, 105 μl, 1.2 mmol) was added to a stirred solution of 5-chloro-6-[(1-methylethyl)oxy]-3-pyridinecarboxylic acid (WO9702244; 215 mg, 1 mmol) in dry dichloromethane (5 ml) followed by DMF (1 drop, catalyst) and the reaction mixture was stirred at room temperature for 1 hour. The solvent was evaporated and the residue dried under vacuum for 30 minutes.

ii) A solution of the crude acid chloride (step i) (1 mmol) in acetonitrile (5 ml) was added dropwise to a stirred solution of 1,1-dimethylethyl 5-[2-(ethyloxy)-2-oxoethyl]-9-[(hydroxyamino)(imino)methyl]-2,3-dihydro-1,4-benzoxazepine-4(5H)-carboxylate (Preparation 34) (393 mg, 1 mmol) and triethylamine (121 mg, 167 μl, 1.2 mmol) in acetonitrile (5 ml) and the mixture stirred at room temperature for 1 hour, then heated under reflux for 72 hours. The reaction mixture was cooled and the solvent evaporated and the residue was purified by chromatography. Eluting with 15-25% ethyl acetate in cyclohexane gave the title compound as pale yellow oil (180 mg).

MS (ES) C₂₈H₃₃³⁵ClN₄O₇ requires 572; found 573 [M+H]⁺.

### Preparation 40

### (9-(5-{5-Chloro-6-[(1-methylethyl)oxy]-3-pyridinyl}-1,2,4-oxadiazol-3-yl)-4-{[(1,1-dimethylethyl)oxy]carbonyl}-2,3,4,5-tetrahydro-1,4-benzoxazepin-5-yl)acetic acid

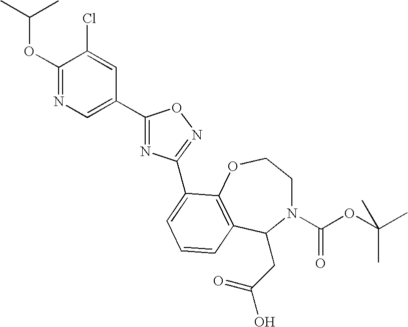

A mixture of 1,1-dimethylethyl 9-(5-{5-chloro-6-[(1-methylethyl)oxy]-3-pyridinyl}-1,2,4-oxadiazol-3-yl)-5-[2-(ethyloxy)-2-oxoethyl]-2,3-dihydro-1,4-benzoxazepine-4(5H)-carboxylate (Preparation 39) (170 mg, 0.30 mmol), 2M sodium hydroxide (5 ml) and ethanol (8 ml) was stirred at 60° C. for 1 hour. The reaction mixture was cooled to room temperature and the ethanol evaporated. The residue was diluted with water (10 ml) and acidified with glacial acetic acid. The mixture was extracted with ethyl acetate (3×5 ml). The combined extracts were dried (Na₂SO₄) and evaporated. The residue was dried under high vacuum to give the title compound as a colourless foam (150 mg). MS (ES) C₂₆H₂₉³⁵ClN₄O₇ requires 544; found 545 [M+H]⁺.

### Preparation 41

### 3-Chloro-4-[(1-methylethyl)oxy]benzoyl chloride

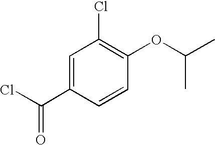

A round bottom flask was charged with 3-chloro-4-[(1-methylethyl)oxy]benzoic acid (Paragos Product List, 10.2 g, 47.5 mmol), dichloromethane (158 ml) and oxalyl chloride (8.29 ml, 95 mmol). The reaction mixture was cooled to 0° C. in an ice/water bath prior to the addition of N,N-dimethylformamide (0.158 ml). The solution was allowed to warm to ambient temperature overnight. The solvent was evaporated to yield the title compound as a cream solid (11.4 g). δH(CDCl3, 400 MHz): 1.44 (6H, d), 4.73 (1H, septet), 6.98 (1H, d), 8.0 (1H, dd), 7.98 (1H, d).

### Preparation 42

### 1,1-dimethylethyl 9-cyano-5-(2-hydroxyethyl)-2,3-dihydro-1,4-benzoxazepine-4(5H)-carboxylate

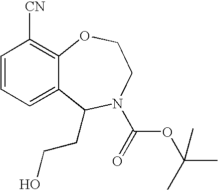

To a solution of 1,1-dimethylethyl 9-cyano-5-[2-(ethyloxy)-2-oxoethyl]-2,3-dihydro-1,4-benzoxazepine-4(5H)-carboxylate (Preparation 33) (282 mg, 0.78 mmol) in ethanol (5 ml) was added lithium borohydride, 2.0M solution in THF (0.587 ml, 1.17 mmol). After stirring at room temperature for 1 hour diethyl ether (5 ml) was added. After 30 minutes lithium borohydride, 2.0M solution in THF (0.500 ml, 1.0 mmol) was added and the reaction mixture stirred at room temperature for 3 days. The suspension was dissolved in methanol (40 ml) and quenched at 0° C. with 2M hydrochloric acid. Most of the solvent was evaporated. The residue was diluted with ethyl acetate, and washed with brine. The organic phase was dried and evaporated to give the title compound (224 mg). MS (ES) C₁₇H₂₂N₂O₄ requires 318 found 319 [M+H]⁺.

### Preparation 43

### 1,1-dimethylethyl 9-cyano-5-[(2E)-4-(ethyloxy)-4-oxo-2-buten-1-yl]-2,3-dihydro-1,4-benzoxazepine-4(5H)-carboxylate

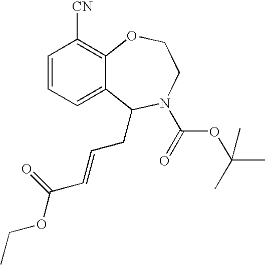

To a solution of oxalyl chloride (777 mg, 0.54 ml, 6.12 mmol) in dichloromethane (25 ml) at −78° C. under nitrogen was added a solution of dimethyl sulfoxide (552 mg, 0.50 ml, 7.07 mmol) in dichloromethane (5 ml) over 5 minutes. After 10 minutes a solution of 1,1-dimethylethyl 9-cyano-5-(2-hydroxyethyl)-2,3-dihydro-1,4-benzoxazepine-4(5H)-carboxylate (Preparation 42) (1.50 g, 4.71 mmol) in dichloromethane (10 ml) was added. The reaction mixture was stirred at −78° C. for 70 minutes then triethylamine (1.43 g, 1.97 ml, 14.1 mmol) was added dropwise over 2 minutes. The mixture was allowed to warm to room temperature. After 40 minutes a solution of ethyl triphenylphosphorylideneacetate (1.97 g, 5.65 mmol) in dichloromethane (20 ml) was added. The reaction mixture was stirred at room temperature for 1 hour. The solvent was evaporated and the residue partitioned between ethyl acetate and brine. The organic phase was separated, dried and evaporated. Purification by chromatography, eluting with 0-50% ethyl acetate in cyclohexane gave the title compound as a colourless oil. (1.76 g). MS (ES) C₂₁H₂₆N₂O₅ requires 386 found 387 [M+H]⁺.

### Preparation 44

### 1,1-dimethylethyl 9-cyano-5-[4-(ethyloxy)-4-oxobutyl]-2,3-dihydro-1,4-benzoxazepine-4(5H)-carboxylate

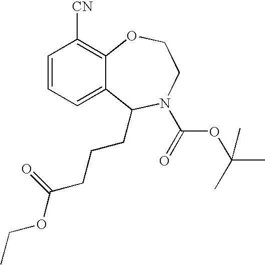

A mixture of 1,1-dimethylethyl 9-cyano-5-[(2E)-4-(ethyloxy)-4-oxo-2-buten-1-yl]-2,3-dihydro-1,4-benzoxazepine-4(5H)-carboxylate (Preparation 43) (780 mg, 2.02 mmol), and 10% palladium on carbon, 50% water paste, (430 mg) in ethanol (30 ml) was stirred under an atmosphere of hydrogen for 90 minutes. The reaction mixture was filtered through ‘celite’ and the filtrate evaporated to give the title compound (680 mg). MS (ES) O₂₁H₂₈N₂O₅ requires 388 found 389 [M+H]⁺.

### Preparation 45

### 1,1-dimethylethyl 5-[4-(ethyloxy)-4-oxobutyl]-9-[(hydroxyamino)(imino)methyl]-2,3-dihydro-1,4-benzoxazepine-4(5H)-carboxylate

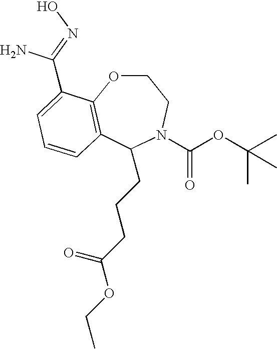

A mixture of 1,1-dimethylethyl 9-cyano-5-[4-(ethyloxy)-4-oxobutyl]-2,3-dihydro-1,4-benzoxazepine-4(5H)-carboxylate (Preparation 44) (750 mg, 1.93 mmol), hydroxylamine hydrochloride (335 mg, 4.83 mmol) and sodium bicarbonate (405 mg, 4.83 mmol) in ethanol (10 ml) was stirred at 70° C. for 24 hours. The reaction mixture was cooled and filtered through ‘celite’. The filtrate was evaporated leaving a colourless gum (814 mg) which was used in preparation 46 without further purification. MS (ES) C₂₁H₃₁N₃O₆ requires 421 found 422 [M+H]⁺.

### Preparation 46

### 1,1-dimethylethyl 9-(5-{3-chloro-4-[(1-methylethyl)oxy]phenyl}-1,2,4-oxadiazol-3-yl)-5-[4-(ethyloxy)-4-oxobutyl]-2,3-dihydro-1,4-benzoxazepine-4(5H)-carboxylate

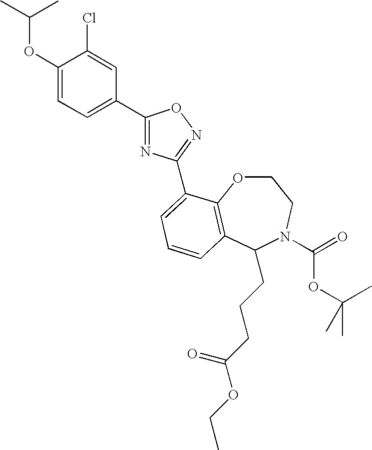

Oxalyl chloride (152 mg, 105 μl, 1.20 mmol) was added to a stirred solution of 3-chloro-4-[(1-methylethyl)oxy]benzoic acid (Paragos Product List, 215 mg, 1.00 mmol) in dry dichloromethane (10 ml) followed by DMF (1 drop) The reaction mixture was stirred at room temperature for 1 hour, then the solvent was evaporated and the residue dried under vacuum for 30 minutes. A solution of the crude acid chloride (1.00 mmol) in acetonitrile (5 ml) was added dropwise to a stirred solution of 1,1-dimethylethyl 5-[4-(ethyloxy)-4-oxobutyl]-9-[(hydroxyamino)(imino)methyl]-2,3-dihydro-1,4-benzoxazepine-4(5H)-carboxylate (Preparation 45) (270 mg, 0.64 mmol) and triethylamine (121 mg, 167 μl, 1.2 mmol) in acetonitrile (5 ml) and stirred at room temperature for 1 hour. The reaction mixture was heated under reflux for 72 hours, then cooled to room temperature and the solvent evaporated. The residue was Purified by chromatography, eluting with 15-25% ethyl acetate in cyclohexane to give the title compound as colourless oil (70 mg). MS (ES) C₃₁H₃₈³⁵ClN₃O₇ requires 599 found 600 [M+H]⁺.

### Preparation 47

### 1,1-dimethylethyl 9-(5-{3-cyano-4-[(1-methylethyl)oxy]phenyl}-1,2,4-oxadiazol-3-yl)-5-[4-(ethyloxy)-4-oxobutyl]-2,3-dihydro-1,4-benzoxazepine-4(5H)-carboxylate

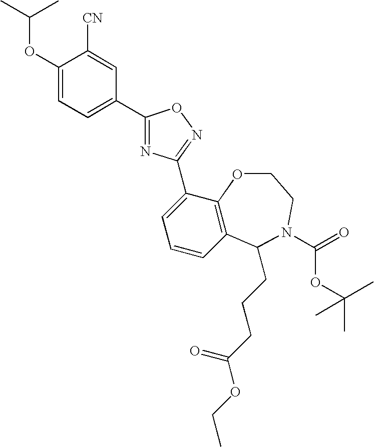

Oxalyl chloride (152 mg, 105 μl, 1.20 mmol) was added to a stirred solution of 3-cyano-4-[(1-methylethyl)oxy]benzoic acid (WO2001002355; 205 mg, 1.00 mmol) in dry dichloromethane (10 ml) followed by DMF (1 drop,) The reaction mixture was stirred at room temperature for 1 hour. The solvent was evaporated and the residue dried under vacuum for 30 minutes. A solution of the crude acid chloride (1.00 mmol) in acetonitrile (5 ml) was added dropwise to a stirred solution of 1,1-dimethylethyl 5-[4-(ethyloxy)-4-oxobutyl]-9-[(hydroxyamino)(imino)methyl]-2,3-dihydro-1,4-benzoxazepine-4(5H)-carboxylate (Preparation 45) (270 mg, 0.64 mmol) and triethylamine (121 mg, 167 μl, 1.20 mmol) in acetonitrile (5 ml) and stirred at room temperature for 1 hour. Then the reaction mixture was heated under reflux for 72 hours then cooled to room temperature and the solvent evaporated. The residue was purified by chromatography, eluting with 15-25% ethyl acetate in cyclohexane to give the title compound as colourless oil (120 mg). MS (ES) C₃₂H₃₈N₄O₇ requires 590 found 591 [M+H]⁺.

### Preparation 48

### 1,1-dimethylethyl 9-(5-{5-chloro-6-[(1-methylethyl)oxy]-3-pyridinyl}-1,2,4-oxadiazol-3-yl)-5-[4-(ethyloxy)-4-oxobutyl]-2,3-dihydro-1,4-benzoxazepine-4(5H)-carboxylate

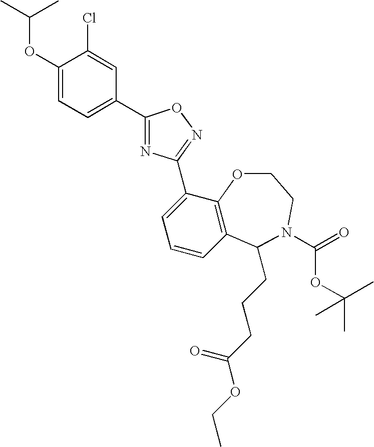

Oxalyl chloride (152 mg, 105 μl, 1.20 mmol) was added to a stirred solution of 5-chloro-6-[(1-methylethyl)oxy]-3-pyridinecarboxylic acid (WO9702244; 215 mg, 1.00 mmol) in dry dichloromethane (10 ml) followed by DMF (1 drop). The reaction mixture was stirred at room temperature for 1 hour. The solvent was evaporated and the residue dried under vacuum for 30 minutes. A solution of the crude acid chloride (1.00 mmol) in acetonitrile (5 ml) was added dropwise to a stirred solution of 1,1-dimethylethyl 5-[4-(ethyloxy)-4-oxobutyl]-9-[(hydroxyamino)(imino)methyl]-2,3-dihydro-1,4-benzoxazepine-4(5H)-carboxylate (Preparation 45) (270 mg, 0.64 mmol) and triethylamine (121 mg, 167 μl, 1.20 mmol) in acetonitrile (5 ml) and stirred at room temperature for 1 hour. The reaction mixture was heated under reflux for 72 hours, then cooled to room temperature and the solvent evaporated. The residue was purified by chromatography, eluting with 15-25% ethyl acetate in cyclohexane to give the title compound as colourless oil which solidified on standing (147 mg). MS (ES) C₃₀H₃₇³⁵ClN₄O₇ requires 600 found 601 [M+H]⁺.

### Preparation 49

### 4-(9-(5-{3-cyano-4-[(1-methylethyl)oxy]phenyl}-1,2,4-oxadiazol-3-yl)-4-{[(1,1-dimethylethyl)oxy]carbonyl}-2,3,4,5-tetrahydro-1,4-benzoxazepin-5-yl)butanoic acid

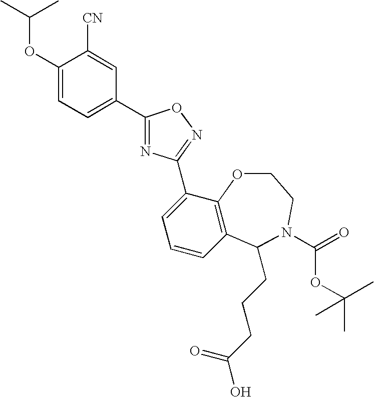

A mixture of 1,1-dimethylethyl 9-(5-{3-cyano-4-[(1-methylethyl)oxy]phenyl}-1,2,4-oxadiazol-3-yl)-5-[4-(ethyloxy)-4-oxobutyl]-2,3-dihydro-1,4-benzoxazepine-4(5H)-carboxylate (Preparation 47) (120 mg, 0.20 mmol), 2M sodium hydroxide (2 ml) and ethanol (2 ml) was stirred at 50° C. for 2 hours. The reaction mixture was cooled to room temperature and the solvent evaporated. The residue was diluted with water (10 ml) and acidified with glacial acetic acid. The mixture was extracted with ethyl acetate (3×5 ml) and the combined organics were dried and evaporated. Trituration of the residue with a mixture of iso-hexane/diethyl ether gave the title compound as a solid (80 mg). MS (ES) C₃₀H₃₄N₄O₇ requires 562 found 563 [M+H]⁺.

### Preparation 50

### 4-(9-(5-{3-chloro-4-[(1-methylethyl)oxy]phenyl}-1,2,4-oxadiazol-3-yl)-4-{[(1,1-dimethylethyl)oxy]carbonyl}-2,3,4,5-tetrahydro-1,4-benzoxazepin-5-yl)butanoic acid

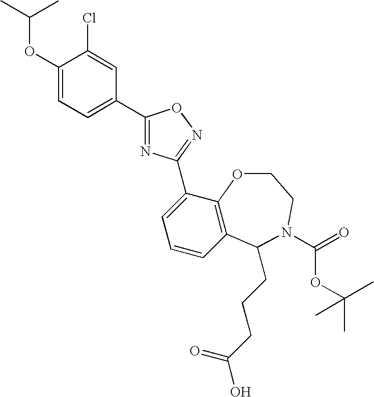

A mixture of 1,1-dimethylethyl 9-(5-{3-chloro-4-[(1-methylethyl)oxy]phenyl}-1,2,4-oxadiazol-3-yl)-5-[4-(ethyloxy)-4-oxobutyl]-2,3-dihydro-1,4-benzoxazepine-4(5H)-carboxylate (Preparation 46) (70 mg, 0.12 mmol), 2M sodium hydroxide (2 ml) and ethanol (2 ml) was stirred at 50° C. for 2 hours. The reaction mixture was cooled to room temperature and the solvent evaporated. The residue was diluted with water (10 ml) and acidified with glacial acetic acid. The mixture was extracted with ethyl acetate (3×5 ml) and the combined organics were dried and evaporated. Trituration of the residue with a mixture of iso-hexane/diethyl ether gave the title compound as a solid (63 mg). MS (ES) C₂₉H₃₄³⁵ClN₃O₇ requires 571 found 572 [M+H]⁺.

### Preparation 51

### 4-(9-(5-{5-chloro-6-[(1-methylethyl)oxy]-3-pyridinyl}-1,2,4-oxadiazol-3-yl)-4-{[(1,1-dimethylethyl)oxy]carbonyl}-2,3,4,5-tetrahydro-1,4-benzoxazepin-5-yl)butanoic acid

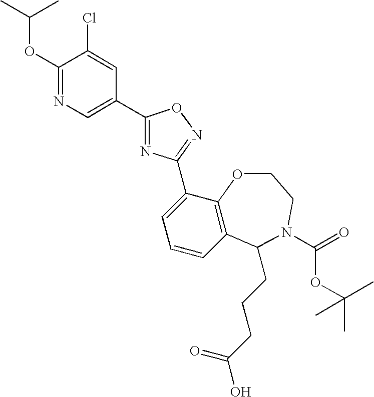

A mixture of 1,1-dimethylethyl 9-(5-{5-chloro-6-[(1-methylethyl)oxy]-3-pyridinyl}-1,2,4-oxadiazol-3-yl)-5-[4-(ethyloxy)-4-oxobutyl]-2,3-dihydro-1,4-benzoxazepine-4(5H)-carboxylate (Preparation 48) (145 mg, 0.24 mmol), 2M sodium hydroxide (2 ml) and ethanol (2 ml) was stirred at 50° C. for 2 hours. The reaction mixture was cooled to room temperature and the solvent evaporated. The residue was diluted with water (10 ml) and acidified with glacial acetic acid. The mixture was extracted with ethyl acetate (3×5 ml) and the combined organics were dried and evaporated. Trituration of the residue with iso-hexane gave the title compound as a solid (147 mg). MS (ES) C₂₈H₃₃³⁵ClN₄O₇ requires 572 found 573 [M+H]⁺.

### Preparation 52

### 1,1-Dimethylethyl 9-cyano-5-(2-oxoethyl)-2,3-dihydro-1,4-benzoxazepine-4(5H)-carboxylate

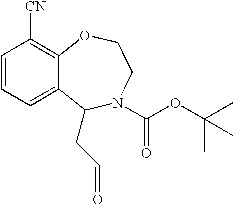

Dimethyl sulphoxide (0.504 g, 6.45 mmol) in dichloromethane (3 ml) was added dropwise to a solution of oxalyl chloride (710 mg, 5.59 mmol) in dichloromethane (40 ml) at −78° C. After 15 mins a solution of 1,1-dimethylethyl 9-cyano-5-(2-hydroxyethyl)-2,3-dihydro-1,4-benzoxazepine-4(5H)-carboxylate (Preparation 42) (1.37 g, 4.30 mmol) in dichloromethane (20 ml) was added over 5 mins and the mixture stirred for 1 h at −78° C., then triethylamine (1.31 g, 12.91 mmol) was added and the mixture stirred for a further 5 mins. The mixture was allowed to warm to room temperature over 45 mins. The solvent was evaporated and the residue partitioned between water and ethyl acetate. The organic extracts were washed with brine and dried (MgSO4). The solvent was evaporated to give the crude title compound as a pale yellow oil which was used crude in the subsequent step

C₁₇H₂₀N₂O₄ requires 316 found 317 [M+H]⁺.

### Preparation 53

### 1,1-Dimethylethyl 9-cyano-5-[(2Z)-3-(methyloxy)-2-propen-1-yl]-2,3-dihydro-1,4-benzoxazepine-4(5H)-carboxylate and 1,1-dimethylethyl 9-cyano-5-[(2E)-3-(methyloxy)-2-propen-1-yl]-2,3-dihydro-1,4-benzoxazepine-4(5H)-carboxylate 1:1

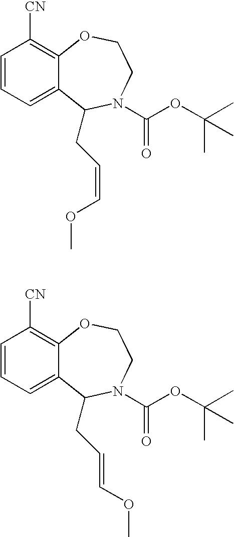

Lithium diisopropylamide (LDA) (0.5M in THF; 5.16 mmol) was added dropwise to a solution of methoxymethyl diphenyl phosphine oxide (1.376 g, 5.59 mmol) in dry THF (20 ml) at 0° C. under nitrogen. The mixture was stirred at 0° C. for 10 mins, before cooling to −78° C. A solution of 1,1-dimethylethyl 9-cyano-5-(2-oxoethyl)-2,3-dihydro-1,4-benzoxazepine-4(5H)-carboxylate (Preparation 52) (1.36 g, 4.30 mmol) in THF (10 ml) was then added drop-wise. The mixture was stirred for 5 mins, the ice bath was removed and the mixture was allowed to warm to room temperature over 40 mins. Saturated ammonium chloride was added and the reaction mixture extracted with diethyl ether. The combined organic extracts were washed with brine, dried (MgSO4) and evaporated to give a pale yellow foam. This material was dissolved in THF (30 ml), then sodium hydride (60%; 430 mg, 4.30 mmol) was added portion wise and the mixture stirred overnight. Methanol (3 ml) was added at 0° C. and the mixture partitioned between ammonium chloride and diethyl ether. The organic extract was washed with brine and dried (MgSO4). The solvent was evaporated and the residue purified by chromatography, eluting with 5-25% ethyl acetate in cyclohexane to give a 1:1 mixture of the title compounds as colourless oil (624 mg). MS (ES) C₁₉H₂₄N₂O₄ requires 344 found 345 [M+H]⁺.

### Preparation 54

### 1,1-Dimethylethyl 9-cyano-5-[3-(methyloxy)-3-oxopropyl]-2,3-dihydro-1,4-benzoxazepine-4(5H)-carboxylate

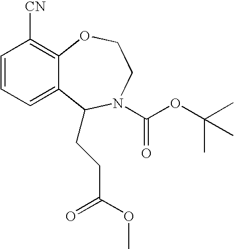

A mixture of 1,1-dimethylethyl 9-cyano-5-[(2Z)-3-(methyloxy)-2-propen-1-yl]-2,3-dihydro-1,4-benzoxazepine-4(5H)-carboxylate and 1,1-dimethylethyl 9-cyano-5-[(2E)-3-(methyloxy)-2-propen-1-yl]-2,3-dihydro-1,4-benzoxazepine-4(5H)-carboxylate 1:1 (Preparation 53) (625 mg, 1.81 mmol) in dichloromethane (10 ml) was added rapidly to a suspension of pyridinium chlorochromate (PCC; 782 mg, 3.63 mmol) in dichloromethane (10 ml) at room temperature. The mixture was allowed to stir overnight, then further PCC (782 mg, 3.63 mmol) was added. The reaction mixture was stirred for a further 24 h, then more PCC (782 mg, 3.63 mmol) was added and the mixture stirred for a further 24 h. The mixture was filtered through Celite®, washing through with dichloromethane. The solvent was evaporated and the residue purified by chromatography, eluting with 5-25% ethyl acetate in cyclohexane to give the title compound as colourless oil (364 mg). MS (ES) C₁₉H₂₄N₂O₅ requires 360 found 361 [M+H]⁺.

### Preparation 55

### 1,1-Dimethylethyl 9-[(hydroxyamino)(imino)methyl]-5-[3-(methyloxy)-3-oxopropyl]-2,3-dihydro-1,4-benzoxazepine-4(5H)-carboxylate

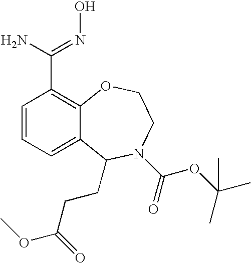

A mixture of 1,1-dimethylethyl 9-cyano-5-[3-(methyloxy)-3-oxopropyl]-2,3-dihydro-1,4-benzoxazepine-4(5H)-carboxylate (Preparation 54) (360 mg, 1.00 mmol), hydroxylamine hydrochloride (174 mg, 2.50 mmol), and sodium bicarbonate (210 mg, 2.50 mmol) in methanol (10 ml) was heated under reflux for 24 hours. A further portion of hydroxylamine hydrochloride (70 mg, 1.00 mmol), and sodium bicarbonate (84 mg, 1.00 mmol) was added and reflux continued for 24 hours. The reaction mixture was cooled and filtered through Celite®. The solvent was evaporated to give the title compound as a colourless solid (393 mg) which was used without further purification. MS (ES) C₁₉H₂₇N₃O₆ requires 393 found 394 [M+H]⁺.

### Preparation 56

### 1,1-Dimethylethyl 9-(5-{3-chloro-4-[(1-methylethyl)oxy]phenyl}-1,2,4-oxadiazol-3-yl)-5-[3-(methyloxy)-3-oxopropyl]-2,3-dihydro-1,4-benzoxazepine-4(5H)-carboxylate

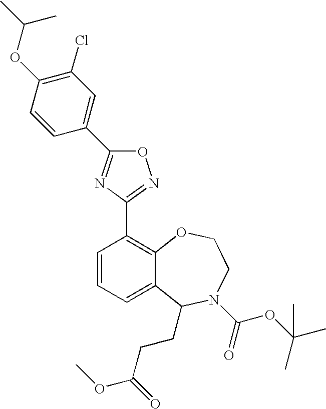

A solution of 1,1-dimethylethyl 9-[(hydroxyamino)(imino)methyl]-5-[3-(methyloxy)-3-oxopropyl]-2,3-dihydro-1,4-benzoxazepine-4(5H)-carboxylate (Preparation 55) (197 mg, 0.50 mmol) in acetonitrile (5 ml) was added slowly to a stirred solution of 3-chloro-4-[(1-methylethyl)oxy]benzoyl chloride (Preparation 41) (117 mg, 0.50 mmol) and triethylamine (61 mg, 84 μl, 0.60 mmol) in acetonitrile (5 ml). The reaction mixture was stirred at room temperature for 1 hour then heated under reflux for 48 hours. The reaction mixture was cooled to room temperature and the solvent evaporated. The residue was purified by chromatography, eluting with 20% ethyl acetate in iso-hexane gave the title compound as a yellow oil (29 mg). MS (ES) C₂₉H₃₄³⁵ClN₃O₇ requires 571 found 572 [M+H]⁺.

### Preparation 57

### 1,1-dimethylethyl 9-(5-{3-cyano-4-[(1-methylethyl)oxy]phenyl}-1,2,4-oxadiazol-3-yl)-5-[3-(methyloxy)-3-oxopropyl]-2,3-dihydro-1,4-benzoxazepine-4(5H)-carboxylate

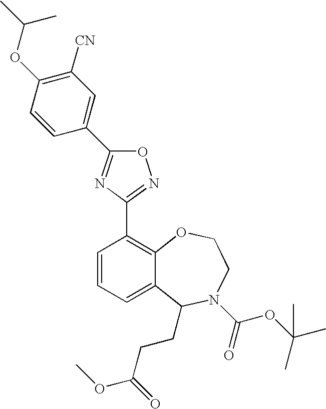

A solution of 1,1-dimethylethyl 9-[(hydroxyamino)(imino)methyl]-5-[3-(methyloxy)-3-oxopropyl]-2,3-dihydro-1,4-benzoxazepine-4(5H)-carboxylate (Preparation 55) (197 mg, 0.50 mmol) in acetonitrile (5 ml) was added slowly to a stirred solution of 3-cyano-4-[(1-methylethyl)oxy]benzoyl chloride (Prepared using the method described for preparation 37) (112 mg, 0.5 mmol) and triethylamine (61 mg, 84 μl, 0.6 mmol) in acetonitrile (5 ml). The reaction mixture was stirred at room temperature for 1 hour then heated under reflux for 48 hours. The reaction mixture was cooled to room temperature and the solvent evaporated. The residue was purified by chromatography, eluting with 20% ethyl acetate in iso-hexane gave the title compound as a yellow oil (41 mg). MS (ES) C₃₀H₃₄N₄O₇ requires 562 found 563 [M+H]⁺.

### Preparation 58

### 3-(9-(5-{3-Chloro-4-[(1-methylethyl)oxy]phenyl}-1,2,4-oxadiazol-3-yl)-4-{[(1,1-dimethylethyl)oxy]carbonyl}-2,3,4,5-tetrahydro-1,4-benzoxazepin-5-yl)propanoic acid

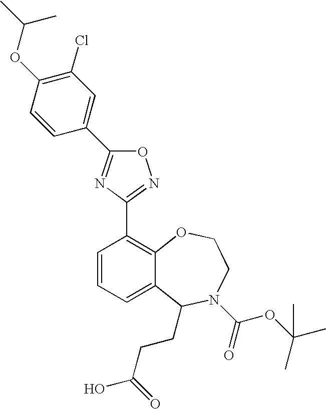

A mixture of 1,1-dimethylethyl 9-(5-{3-chloro-4-[(1-methylethyl)oxy]phenyl}-1,2,4-oxadiazol-3-yl)-5-[3-(methyloxy)-3-oxopropyl]-2,3-dihydro-1,4-benzoxazepine-4(5H)-carboxylate (Preparation 56) (29 mg, 0.05 mmol), 2M sodium hydroxide (2 ml), and ethanol (2 ml) was stirred at 60° C. for 2 hours. The reaction mixture was cooled and the ethanol was evaporated. The residue was diluted with water (5 ml) and acidified with glacial acetic acid. The solution was extracted with ethyl acetate (2×5 ml). The combined organics were dried and evaporated to give the title compound as a brown gum (24 mg) which was used without further purification MS (ES) C₂₈H₃₂³⁵ClN₃O₇ requires 557 found 558 [M+H]⁺.

### Preparation 59

### 3-(9-(5-{3-Cyano-4-[(1-methylethyl)oxy]phenyl}-1,2,4-oxadiazol-3-yl)-4-{[(1,1-dimethylethyl)oxy]carbonyl}-2,3,4,5-tetrahydro-1,4-benzoxazepin-5-yl)propanoic acid

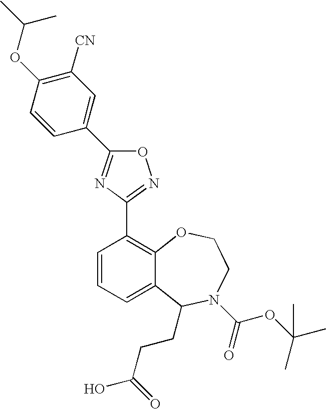

A mixture of 1,1-dimethylethyl 9-(5-{3-cyano-4-[(1-methylethyl)oxy]phenyl}-1,2,4-oxadiazol-3-yl)-5-[3-(methyloxy)-3-oxopropyl]-2,3-dihydro-1,4-benzoxazepine-4(5H)-carboxylate (Preparation 57) (41 mg, 0.07 mmol), 2M sodium hydroxide (2 ml), and ethanol (2 ml) was stirred at 60° C. for 2 hours. The reaction mixture was cooled and the ethanol was evaporated. The residue was diluted with water (5 ml) and acidified with glacial acetic acid. The solution was extracted with ethyl acetate (2×5 ml). The combined organics were dried and evaporated to give the title compound as a yellow gum (36 mg) which was used without further purification. MS (ES) C₂₉H₃₂N₄O₇ requires 548 found 549 [M+H]⁺.

### EXAMPLE 1

### 3-[7-(5-{3-chloro-4-[(1-methylethyl)oxy]phenyl}-1,2,4-oxadiazol-3-yl)-2,3,4,5-tetrahydro-1H-2-benzazepin-1-yl]propanamide

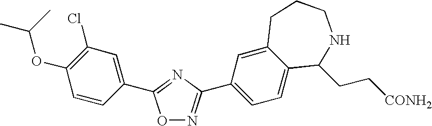

A solution of 1,1-dimethylethyl 7-(5-{3-chloro-4-[(1-methylethyl)oxy]phenyl}-1,2,4-oxadiazol-3-yl)-1-[3-(methyloxy)-3-oxopropyl]-1,3,4,5-tetrahydro-2H-2-benzazepine-2-carboxylate (0.050 g, 0.088 mmol) in 7M ammonia in methanol (5 ml) was stirred at room temperature for 7 days. The mixture was evaporated, re-dissolved in DCM (1 ml) and trifluoroacetic acid (1.000 ml, 13 mmol), then stirred at room temperature for 30 min. The mixture was evaporated, and then purified by MDAP. The fraction containing product was evaporated to ˜10 ml, basified with 2M aq sodium hydroxide and extracted with ethyl acetate (30 ml). The organics were dried (magnesium sulphate), evaporated and the residue triturated with ether to leave a white solid (18 mg). m/z (ES+) 455 [M+H]⁺. ¹H NMR (CDCl₃) δ 1.45 (6H, d), 1.65 (1H, m), 1.82 (1H, m), 2.17 (1H, m), 2.28 (1H, m), 2.47-2.55 (2H, m), 3.07-3.13 (3H, m), 3.36 (1H, m), 3.95 (1H, m), 4.72 (1H, m), 5.3 (1H, br s), 6.1 (1H, br s), 7.06 (1H, d), 7.32 (1H, d), 7.92-7.94 (2H, m), 8.06 (1H, dd), 8.24 (1H, d).

### EXAMPLE 2

### 3-[7-(5-{3-Chloro-4-[(1-methylethyl)oxy]phenyl}-1,2,4-oxadiazol-3-yl)-2,3,4,5-tetrahydro-1H-2-benzazepin-1-yl]propanoic acid hydrochloride

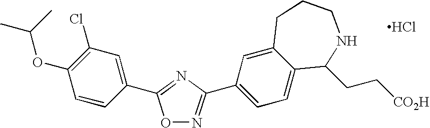

3-(7-(5-{3-Chloro-4-[(1-methylethyl)oxy]phenyl}-1,2,4-oxadiazol-3-yl)-2-{[(1,1-dimethylethyl)oxy]carbonyl}-2,3,4,5-tetrahydro-1H-2-benzazepin-1-yl)propanoic acid (0.086 g, 0.155 mmol) was dissolved in a solution of trifluoroacetic acid (1.0 ml, 13.0 mmol) and DCM (1 ml) and stirred at room temperature for 30 min then evaporated to dryness. Purification by MDAP left a residue that was redissolved in DCM (5 ml) before 1M HCl in ether (1 ml) was added. The mixture was evaporated, then suspended in ether and filtered to leave a white solid (21 mg). m/z (ES+) 456 [M+H]⁺. ¹H NMR (d₄-MeOD) δ 1.42 (6H, d), 2.06 (2H, m), 2.39 (1H, m), 2.48 (2H, m), 2.55 (1H, m), 3.24 (2H, m), 3.46 (1H, m), 3.49 (1H, m), 4.74 (1H, m), 4.84 (1H, m), 7.32 (1H, d), 7.56 (1H, d), 8.07-8.13 (3H, m), 8.21 (1H, d).

### EXAMPLE 3

### 3-[7-(5-{3-Chloro-4-[(1-methylethyl)oxy]phenyl}-1,2,4-oxadiazol-3-yl)-2,3,4,5-tetrahydro-1H-2-benzazepin-1-yl]-1-propanol hydrochloride

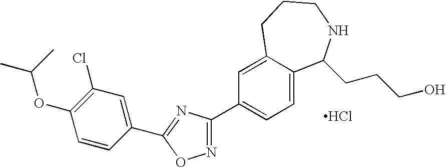

1M Lithium aluminium hydride in THF (0.149 ml, 0.149 mmol) was added to a solution of methyl 3-[7-(5-{3-chloro-4-[(1-methylethyl)oxy]phenyl}-1,2,4-oxadiazol-3-yl)-2,3,4,5-tetrahydro-1H-2-benzazepin-1-yl]propanoate (0.07 g, 0.149 mmol) in THF (3 ml) and the resulting mixture stirred at room temperature for 30 min. A 2M aq sodium hydroxide (10 ml) was added, then the mixture extracted into ethyl acetate (20 ml) and the organic phase dried (magnesium sulphate), evaporated and purified by MDAP. The desired fractions were combined, evaporated, azeotroped with ethanol (20 ml) and the residue re-dissolved in DCM (4 ml). A solution of 1M HCl in ether (1 ml) was added, then the mixture evaporated and triturated with ether to leave a white solid (31 mg). m/z (ES+) 442 [M+H]⁺. ¹H NMR (d₄-MeOD) δ 1.42 (6H, d), 1.55-1.75 (2H, m), 2.06 (2H, m), 2.19 (1H, m), 2.39 (1H, m), 3.24 (2H, m), 3.45-3.55 (2H, m), 3.57 (2H, m), 4.68 (1H, m), 4.85 (1H, m), 7.32 (1H, d), 7.59 (1H, d), 8.07-8.13 (3H, m), 8.21 (1H, d).

### EXAMPLE 4

### [7-(5-{3-Chloro-4-[(1-methylethyl)oxy]phenyl}-1,2,4-oxadiazol-3-yl)-2,3,4,5-tetrahydro-1,4-benzoxazepin-3-yl]methanol hydrochloride

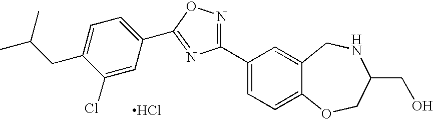

1,1-Dimethylethyl 7-(5-{3-chloro-4-[(1-methylethyl)oxy]phenyl}-1,2,4-oxadiazol-3-yl)-3-(hydroxymethyl)-2,3-dihydro-1,4-benzoxazepine-4(5H)-carboxylate (0.024 g, 0.047 mmol) was dissolved in 4M HCl in dioxane (1.0 ml, 4.0 mmol) and stirred at room temperature for 1 h. The mixture was evaporated and the residue triturated with ether to leave a white solid (20 mg). m/z (ES+) 416 [M+H]⁺. ¹H NMR (d₆-DMSO) δ 1.36 (6H, d), 3.70-3.75 (3H, m), 4.07 (1H, m), 4.46-4.57 (3H, m), 4.89 (1H, m), 5.52 (1H, m), 7.28 (1H, d), 7.46 (1H, d), 8.05 (1H, dd), 8.11 (1H, dd), 8.19 (2H, m), 9.66 (2H, br s).

### EXAMPLE 5

### 7-(5-{3-Chloro-4-[(1-methylethyl)oxy]phenyl}-1,2,4-oxadiazol-3-yl)-2,3,4,5-tetrahydro-1,4-benzoxazepine-3-carboxylic acid hydrochloride

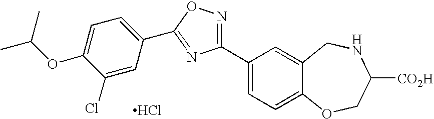

To a solution of 7-(5-{3-chloro-4-[(1-methylethyl)oxy]phenyl}-1,2,4-oxadiazol-3-yl)-4-{[(1,1-dimethylethyl)oxy]carbonyl}-2,3,4,5-tetrahydro-1,4-benzoxazepine-3-carboxylic acid (0.069 g, 0.13 mmol) in 1,4-dioxane (1 ml) was added 4M HCl in dioxane (4.0 ml, 16.0 mmol) and the resulting solution stirred at room temperature for 1.5 hours. The mixture was evaporated to dryness and triturated with ether to leave a white solid (39 mg). m/z (ES+) 430 [M+H]⁺. ¹H NMR (d₆-DMSO) δ 1.37 (6H, d), 4.39-4.56 (4H, m), 4.65 (1H, m), 4.89 (1H, m), 7.24 (1H, d), 7.45 (1H, d), 8.02 (1H, dd), 8.11 (1H, dd), 8.14 (1H, d), 8.18 (1H, d).

### EXAMPLE 6

### 3-[7-(5-{3-Chloro-4-[(1-methylethyl)oxy]phenyl}-1,2,4-oxadiazol-3-yl)-2,3,4,5-tetrahydro-1,4-benzoxazepin-3-yl]propanoic acid hydrochloride

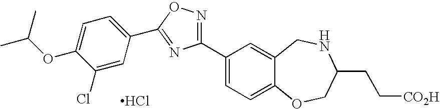

3-(7-(5-{3-Chloro-4-[(1-methylethyl)oxy]phenyl}-1,2,4-oxadiazol-3-yl)-4-{[(1,1-dimethylethyl)oxy]carbonyl}-2,3,4,5-tetrahydro-1,4-benzoxazepin-3-yl)propanoic acid (0.101 g, 0.181 mmol) was dissolved in 4M HCl in dioxane (3.0 ml, 12.0 mmol) and stirred at room temperature for 1 h during which time a solid formed. This was filtered then washed with dioxane and ether to leave a white solid (55 mg). m/z (ES+) 458 [M+H]⁺. ¹H NMR (d₄-MeOD) δ 1.42 (6H, d), 2.00 (2H, m), 2.61 (2H, m), 3.86 (1H, m), 4.02 (1H, m), 4.55 (2H, s), 4.59 (1H, m) 4.84 (1H, m), 7.30 (2H, m), 8.11 (1H, dd), 8.15 (1H, dd), 8.20 (2H, m).

### EXAMPLE 7

### [9-(5-{3-Chloro-4-[(1-methylethyl)oxy]phenyl}-1,2,4-oxadiazol-3-yl)-2,3,4,5-tetrahydro-1,4-benzoxazepin-5-yl]acetic acid hydrochloride

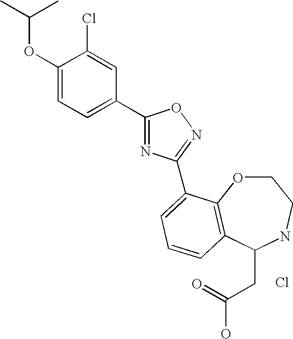

4.0M hydrogen chloride in dioxan (2 ml) was added to a solution of (9-(5-{3-chloro-4-[(1-methylethyl)oxy]phenyl}-1,2,4-oxadiazol-3-yl)-4-{[(1,1-dimethylethyl)oxy]carbonyl}-2,3,4,5-tetrahydro-1,4-benzoxazepin-5-yl)acetic acid, sodium salt (Preparation 36) (90 mg, 0.17 mmol) in dioxan (1 ml). The reaction mixture was stirred at room temperature overnight. Diethyl ether (10 ml) was added and the mixture stirred for 10 minutes. The precipitate was filtered off washed with diethyl ether and dried to give the title compound as a colourless solid (70 mg). δH (400 MHz, d₆DMSO) 1.37 (6H, d), 3.1-3.7 (4H, m) (includes water peak), 4.16-4.27 (1H, m), 4.34-4.45 (1H, m), 4.89 (1H, m), 4.97-5.03 (1H, m), 7.35-7.41 (1H, dd), 7.46 (1H, d), 7.61 (1H, d), 7.96 (1H, d), 8.11 (1H, d), 8.18 (1H, s), 10.00 (2H, br s), 12.80 (1H, br s). MS (ES) C₂₂H₂₂³⁵ClN₃O₅ requires 443; found 444 [M+H]⁺.

### EXAMPLE 8

### [9-(5-{3-cyano-4-[(1-methylethyl)oxy]phenyl}-1,2,4-oxadiazol-3-yl)-2,3,4,5-tetrahydro-1,4-benzoxazepin-5-yl]acetic acid

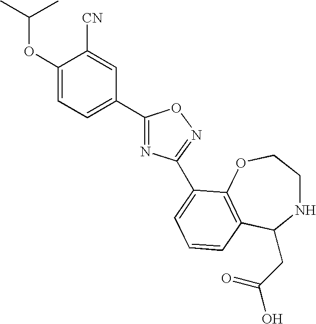

4.0M hydrogen chloride in dioxan (2 ml, 8 mmol) was added to a stirred solution of (9-(5-{3-cyano-4-[(1-methylethyl)oxy]phenyl}-1,2,4-oxadiazol-3-yl)-4-{[(1,1-dimethylethyl)oxy]carbonyl}-2,3,4,5-tetrahydro-1,4-benzoxazepin-5-yl)acetic acid (Preparation 38) (150 mg, 0.28 mmol) in dry dioxan (2 ml) and the mixture was stirred at room temperature for 4 hours. Diethyl ether (10 ml) was added to give a colourless solid. The solid was filtered off and washed with diethyl ether, then purified by MDAP to give the product as a colourless solid (78 mg). δH (400 MHz, d₆DMSO) 1.39 (6H, d), 2.66-2.74 (1H, m), 2.77-2.86 (1H, m), 2.98-3.04 (1H, m), 3.94 (1H, m), 4.09-4.15 (1H, m), 4.40-4.47 (1H, m), 4.98 (1H, m), 7.26 (1H, dd), 7.44 (1H, d), 7.56 (1H, d), 7.80 (1H, d), 8.40 (1H, m), 8.51 (1H, m). MS (ES) C₂₃H₂₂N₄O₅ requires 434; found 435 [M+H]⁺.

### EXAMPLE 9

### [9-(5-{5-Chloro-6-[(1-methylethyl)oxy]-3-pyridinyl}-1,2,4-oxadiazol-3-yl)-2,3,4,5-tetrahydro-1,4-benzoxazepin-5-yl]acetic acid

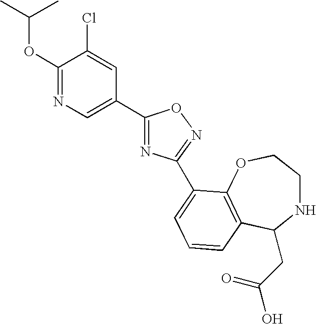

4.0M hydrogen chloride in dioxan (2 ml, 8 mmol) was added to a stirred solution of (9-(5-{5-chloro-6-[(1-methylethyl)oxy]-3-pyridinyl}-1,2,4-oxadiazol-3-yl)-4-{[(1,1-dimethylethyl)oxy]carbonyl}-2,3,4,5-tetrahydro-1,4-benzoxazepin-5-yl)acetic acid (Preparation 40) (250 mg, 0.46 mmol) in dry dioxan (2 ml) and stirred at room temperature for 4 hours. Diethyl ether (10 ml) was added to give a colourless solid which was filtered off and washed with diethyl ether. The solid was purified by MDAP to give the title compound as a colourless solid (169 mg). δH (400 MHz, d₆DMSO) 1.39 (6H, d), 2.66-2.73 (1H, m), 2.77-2.85 (1H, m), 3.07-3.14 (1H, m), 3.93-4.00 (1H, m), 4.08-4.16 (1H, m), 4.40-4.06 (1H, m), 5.45 (1H, m), 7.26 (1H, dd), 7.44 (1H, m), 7.80 (1H, m), 8.54 (1H, d), 8.93 (1H, d). MS (ES) C₂₁H₂₁³⁵ClN₄O₅ requires 444; found 445 [M+H]⁺.

### EXAMPLE 10

### 4-[9-(5-{3-cyano-4-[(1-methylethyl)oxy]phenyl}-1,2,4-oxadiazol-3-yl)-2,3,4,5-tetrahydro-1,4-benzoxazepin-5-yl]butanoic acid hydrochloride salt

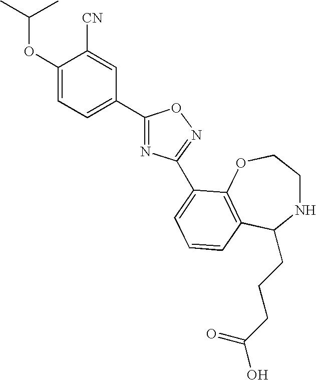

4M HCl in dioxan (1 ml) was added to a stirred solution of 4-(9-(5-{3-cyano-4-[(1-methylethyl)oxy]phenyl}-1,2,4-oxadiazol-3-yl)-4-{[(1,1-dimethylethyl)oxy]carbonyl}-2,3,4,5-tetrahydro-1,4-benzoxazepin-5-yl)butanoic acid (Preparation 49) (80 mg, 0.14 mmol) in dioxan (2 ml). The reaction mixture was stirred at room temperature for 2 hours. Further 4M HCl in dioxan (1 ml) was added and the mixture stirred at room temperature for 1 hour. The solvent was evaporated and dry diethyl ether (2 ml) was added to the residue and the mixture stirred for 30 minutes. The supernatant liquid was decanted off and dry diethyl ether (2 ml) added to the residue. After stirring for 30 minutes the solid was filtered off to give the title compound as a colourless solid (37 mg)

δH (400 MHz, d₆DMSO) 1.20-1.30 (1H, m), 1.39 (6H, d), 1.50-1.60 (1H, m), 2.00-2.15 (2H, m), 2.30 (2H, t), 3.35-3.45 (1H, m) 3.60-3.75 (1H, m), 4.00-4.10 (1H, m), 4.45-4.55 (1H, m), 4.65-4.70 (1H, m), 4.95-5.05 (1H, m), 7.35 (1H, dd), 7.55 (1H, d), 7.70 (1H, d), 7.95 (1H, d), 8.45 (1H, m), 8.50 (1H, m), 9.50-9.80 (2H, br m), 12.1 (1H, br s). MS (ES) C₂₅H₂₆N₄O₅ requires 462 found 463 [M+H]⁺.

### EXAMPLE 11

### 4-[9-(5-{5-chloro-6-[(1-methylethyl)oxy]-3-pyridinyl}-1,2,4-oxadiazol-3-yl)-2,3,4,5-tetrahydro-1,4-benzoxazepin-5-yl]butanoic acid hydrochloride

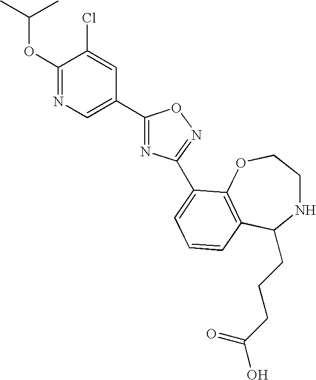

4M HCl in dioxan (1 ml) was added to a stirred solution of 4-(9-(5-{5-chloro-6-[(1-methylethyl)oxy]-3-pyridinyl}-1,2,4-oxadiazol-3-yl)-4-{[(1,1-dimethylethyl)oxy]carbonyl}-2,3,4,5-tetrahydro-1,4-benzoxazepin-5-yl)butanoic acid (Preparation 51) (100 mg, 0.17 mmol) in dioxan (2 ml). The reaction mixture was stirred at room temperature for 2 hours. Further 4M HCl in dioxan (1 ml) was added and the mixture stirred at room temperature for 1 hour. The solvent was evaporated and dry diethyl ether (2 ml) was added to the residue and the mixture stirred for 30 minutes. The supernatant liquid was decanted off and dry diethyl ether (2 ml) added to the residue. After stirring for 30 minutes the solid was filtered off to give the title compound as a colourless solid (21 mg). δH (400 MHz, d₆DMSO) 1.20-1.30 (1H, m), 1.40 (6H, d), 1.50-1.60 (1H, m), 1.95-2.05 (2H, m), 2.30 (2H, t), 3.35-3.45 (1H, m) 3.60-3.72 (1H, m), 4.00-4.10 (1H, m), 4.48-4.56 (1H, m), 4.65-4.70 (1H, m), 5.40-5.50 (1H, m), 7.40 (1H, dd), 7.68-7.72 (1H, m), 7.94-7.99 (1H, m), 8.55 (1H, d), 8.94 (1H, d), 9.40-9.80 (2H, br m), 12.1 (1H, br s). MS (ES) C₂₃H₂₅³⁵ClN₄O₆ requires 472 found 473 [M+H]⁺.

### EXAMPLE 12

### 4-[9-(5-{3-chloro-4-[(1-methylethyl)oxy]phenyl}-1,2,4-oxadiazol-3-yl)-2,3,4,5-tetrahydro-1,4-benzoxazepin-5-yl]butanoic acid hydrochloride

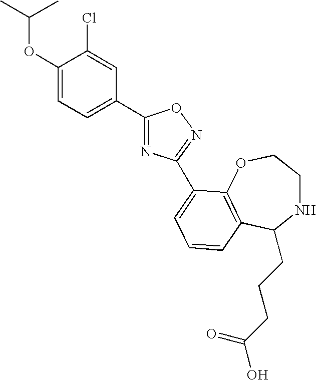

4M HCl in dioxan (1 ml) was added to a stirred solution of 4-(9-(5-{3-chloro-4-[(1-methylethyl)oxy]phenyl}-1,2,4-oxadiazol-3-yl)-4-{[(1,1-dimethylethyl)oxy]carbonyl}-2,3,4,5-tetrahydro-1,4-benzoxazepin-5-yl)butanoic acid (preparation 50) (60 mg, 0.10 mmol) in dioxan (2 ml). The reaction mixture was stirred at room temperature for 2 hours. Further 4M HCl in dioxan (1 ml) was added and stirring at room temperature continued for 1 hour. The solvent was evaporated and dry diethyl ether (2 ml) was added to the residue and the mixture stirred for 30 minutes. The supernatant liquid was decanted off and dry diethyl ether (2 ml) added to the residue. After stirring for 30 minutes the solid was filtered off to give the title compound as a colourless solid (9 mg). δH (400 MHz, d₄ MeOH) 1.42 (6H, d), 1.43-1.55 (1H, m), 1.65-1.75 (1H, m), 2.10-2.20 (1H, m), 2.25-2.35 (1H, m), 2.40 (2H, t), 3.50-3.60 (1H, m) 3.80-3.92 (1H, m), 4.10-4.20 (1H, m), 4.55-4.64 (1H, m), 4.68-4.77 (1H, m), 4.80-4.90 (1H, m) (partially obscured by H₂0), 7.32 (1H, d), 7.38-7.44 (1H, dd), 7.62-7.68 (1H, m), 8.07-8.14 (2H, m), 8.20 (1H, s). MS (ES) C₂₄H₂₆³⁵ClN₄O₅ requires 471 found 472 [M+H]⁺.

### EXAMPLE 13

### 3-[9-(5-{3-Chloro-4-[(1-methylethyl)oxy]phenyl}-1,2,4-oxadiazol-3-yl)-2,3,4,5-tetrahydro-1,4-benzoxazepin-5-yl]propanoic acid, formic acid salt

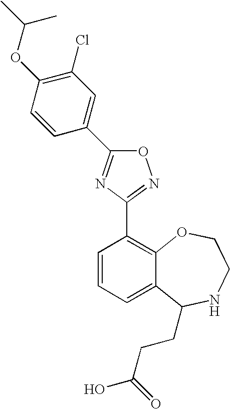

4M HCl in dioxan (1 ml) was added to a solution of 3-(9-(5-{3-chloro-4-[(1-methylethyl)oxy]phenyl}-1,2,4-oxadiazol-3-yl)-4-{[(1,1-dimethylethyl)oxy]carbonyl}-2,3,4,5-tetrahydro-1,4-benzoxazepin-5-yl)propanoic acid (Preparation 58) (25 mg, 0.04 mmol) in dry dioxan (1 ml). The reaction mixture was stirred at room temperature for 1 hour. The solvent was evaporated and the residue triturated under diethyl ether to give a gum which was purified by MDAP to give the title compound as a colourless glass (8 mg).

δH (400 MHz, d₄ MeOH) 1.42 (6H, d), 2.30-2.52 (4H, m), 3.52-3.59 (1H, m), 3.76-3.84 (1H, m), 4.18-4.27 (1H, m), 4.42-4.50 (1H, m), 4.69-4.75 (1H, m), 4.80-4.87 (1H, m), 7.31 (1H, d), 7.38-7.43 (1H, dd), 7.58 (1H, d), 8.05-8.13 (2H, m), 8.19-8.21 (2H, m). MS (ES) C₂₃H₂₄³⁵ClN₃O₇ requires 457 found 458 [M+H]⁺.

### EXAMPLE 14

### 3-[9-(5-{3-cyano-4-[(1-methylethyl)oxy]phenyl}-1,2,4-oxadiazol-3-yl)-2,3,4,5-tetrahydro-1,4-benzoxazepin-5-yl]propanoic acid, formic acid salt

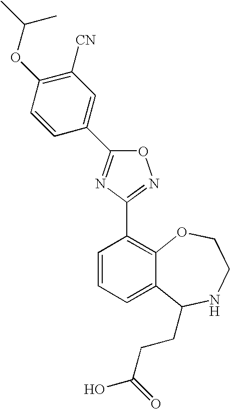

4M HCl in dioxan (1 ml) was added to a solution of 3-(9-(5-{3-cyano-4-[(1-methylethyl)oxy]phenyl}-1,2,4-oxadiazol-3-yl)-4-{[(1,1-dimethylethyl)oxy]carbonyl}-2,3,4,5-tetrahydro-1,4-benzoxazepin-5-yl)propanoic acid (Preparation 59) (40 mg, 0.07 mmol) in dry dioxan (1 ml). The reaction mixture was stirred at room temperature for 2 hours. The solvent was evaporated and the residue triturated under diethyl ether followed by purification by MDAP to give the title compound as a colourless glass (13 mg).

δH (400 MHz, d₄MeOH) 1.45 (6H, d), 2.30-2.42 (3H, m), 2.47-2.55 (1H, m), 3.52-3.58 (1H, m), 3.80-3.88 (1H, m), 4.16-4.25 (1H, m), 4.47-4.55 (1H, m), 4.71-4.78 (1H, m), 4.92-5.00 (1H, m (part obscured by solvent)), 7.38-7.46 (2H, m), 7.57-7.61 (1H, m), 8.07-8.11 (1H, m), 8.15-8.20 (1H, br s), 8.39-8.44 (1H, m), 8.46 (1H, s). MS (ES) C₂₄H₂₄N₄O₅ requires 448 found 449 [M+H]⁺.

### EXAMPLE 15

### [9-(5-{3-Chloro-4-[(1-methylethyl)oxy]phenyl}-1,2,4-oxadiazol-3-yl)-4-methyl-2,3,4,5-tetrahydro-1,4-benzoxazepin-5-yl]acetic acid

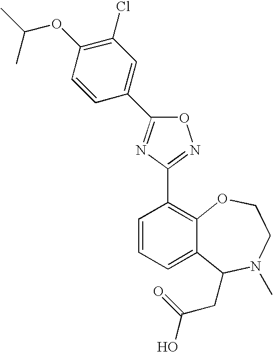

A mixture of ethyl [9-(5-{3-chloro-4-[(1-methylethyl)oxy]phenyl}-1,2,4-oxadiazol-3-yl)-4-methyl-2,3,4,5-tetrahydro-1,4-benzoxazepin-5-yl]acetate (Preparation 61) (75 mg, 0.15 mmol), ethanol (0.5 ml) and 2M sodium hydroxide (1 ml) was stirred at room temperature for 2 hours. The solvent was evaporated and the residue was diluted with water (5 ml) and washed with diethyl ether (2 ml). The aqueous phase was neutralised with glacial acetic acid and extracted with ethyl acetate (2×3 ml). The combined organic extracts were dried and evaporated. The residue was dissolved in ethyl acetate (1 ml) and treated with 1M hydrogen chloride in diethyl ether (1 ml). The solvent was evaporated and the residue triturated under diethyl ether to give the title compound as a colourless solid (40 mg)

δH (400 MHz, d₆DMSO) 1.37 (6H, d), 2.78 (3H, br. s), 2.91-4.09 (4H, m) (includes water peak), 4.29 (1H, t), 4.49 (1H, d), 4.89 (1H, spt), 4.98 (1H, br. s), 7.39 (1H, t), 7.45 (1H, d), 7.66 (1H, d), 8.00 (1H, d), 8.11 (1H, d), 8.18 (1H, s), 11.94 (1H, br s), 12.38 (1H, br s). MS (ES) C₂₃H₂₄³⁵ClN₃O₆ requires 457; found 458 [M+H]⁺.

### Membrane Preparation for S1P1 GTPγS Assay

For membrane preparations all steps were performed at 4° C. Rat hepatoma cells stably expressing the human S1P1 receptor or Rat Basophilic Leukaemia cells (RBL) stably expressing human S1P3 receptor were grown to 80% confluency before being harvested into 10 ml Phospho-Buffered Saline (PBS) and centrifuged at 1200 rpm for 5 minutes. After removal of the supernatant, the pellet was re-suspended and cells were homogenised within a glass Waring blender for 2 bursts of 15 secs in 200 mls of buffer (50 mM HEPES, 1 mM leupeptin, 25 μg/ml bacitracin, 1 mM EDTA, 1 mM PMSF, 2 μM pepstatin A). The blender was plunged into ice for 5 mins after the first burst and 10-40 mins after the final burst to allow foam to dissipate. The material was then spun at 500 g for 20 mins and the supernatant spun for 36 mins at 48,000 g. The pellet was resuspended in the same buffer as above but without PMSF and pepstatin A. The material was then forced through a 0.6 mm needle, made up to the required volume, (usually ×4 the volume of the original cell pellet), aliquoted and stored frozen at −80° C.

### Alternative Membrane Preparation for S1P1 GTPγS Assay

All steps were performed at 4° C. Cells were homogenised within a glass Waring blender for 2 bursts of 15 secs in 200 mls of buffer (50 mM HEPES, 1 mM leupeptin, 25 μg/ml bacitracin, 1 mM EDTA, 1 mM PMSF, 2 μM pepstatin A). The blender was plunged into ice for 5 mins after the first burst and 10-40 mins after the final burst to allow foam to dissipate. The material was then spun at 500 g for 20 mins and the supernatant spun for 36 mins at 48,000 g. The pellet was resuspended in the same buffer as above but without PMSF and pepstatin A. The material was then forced through a 0.6 mm needle, made up to the required volume, (usually ×4 the volume of the original cell pellet), aliquoted and stored frozen at −80° C.

### S1P1 GTPγS Assay

Human S1P1 rat hepatoma membranes (1.5 μg/well) were adhered to a wheatgerm agglutinin (WGA)-coated scintillation proximity assay (SPA) beads (0.125 mg/well) in assay buffer (HEPES 20 mM, MgCl₂ 10 mM, NaCl 100 mM and pH adjusted to 7.4 using KOH 5M, GDP 10 μM FAC (final assay concentration) and saponin 90 μg/ml FAC was also added).

After 30 minutes pre-coupling on ice the bead and membrane suspension was dispensed into a white Greiner polypropylene LV384-well plate (5 μl/well), containing 0.1 μl of the compound. 5 μl/well [³⁵S]-GTPγS (0.5 nM final radioligand conc) made up in assay buffer was then added to agonist plates. The final assay cocktail (10.1 μl) was then centrifuged at 1000 rpm for 5 minutes then read immediately on a Viewlux reader.

All test compounds were dissolved in DMSO at a concentration of 10 mM and were prepared in 100% DMSO using a 1 in 4 dilution step to provide 11 point dose response curves. The dilutions were transferred to the assay plates ensuring that the DMSO concentration was constant across the plate for all assays.

All data was normalized to the mean of 16 high and 16 low control wells on each plate. A four parameter curve fit was then applied.

### Alternative Method for S1P1 GTPγS Assay

S₁P₁ expressing RH7777 membranes (1.5 μg/well) membranes (1.5 μg/well) were homogenised by passing through a 23G needle. These were then adhered to WGA-coated SPA beads (0.125 mg/well) in assay buffer (HEPES 20 mM, MgCl₂ 10 mM, NaCl 100 mM and pH adjusted to 7.4 using KOH 5M). GDP 10 μM FAC and saponin 90 μg/ml FAC were also added

After 30 minutes precoupling on ice, the bead and membrane suspension was dispensed into white Greiner polypropylene LV 384-well plates (5 μl/well), containing 0.1 μl of compound. 5 μl/well [³⁵S]-GTPγS (0.5 nM for S₁P₁ or 0.3 nM for S₁P₃ final radioligand concentration) made in assay buffer was then added to the plates. The final assay cocktail (10.1 μl) was then sealed, spun on a centrifuge, then read immediately on a Viewlux instrument.

Exemplified compounds of the invention had a pEC50>5. Examples 4 to 7 and 9 to 14 had a pEC50 of >7. Examples 6 and 12 to 14 had a pEC50>8.

### S1P3

S1P3 membranes from rat basophilic leukaemia cells (RBL-2H3)(1.5 μg/well) were adhered to WGA-coated SPA beads (0.125 mg/well) in assay buffer (HEPES 20 mM, MgCl₂ 3 mM, NaCl 100 mM and pH adjusted to 7.4 using KOH 5M), GDP 10 μM FAC and saponin 90 μg/ml FAC was also added).

After 30 minutes pre-coupling on ice the bead and membrane suspension was dispensed into a white Greiner polypropylene LV384-well plate (5 μl/well), containing 0.1 μl of the compound. 5 μl/well [³⁵S]-GTPγS (0.5 nM final radioligand conc) made up in assay buffer was then added to agonist plates. The final assay cocktail (10.1 μl) was centrifuged at 1000 rpm for 5 minutes then read immediately on a Viewlux reader.

All test compounds were dissolved in DMSO at a concentration of 10 mM and were prepared in 100% DMSO using a 1 in 4 dilution step to provide 11 point dose response curves. The dilutions were transferred to the assay plates ensuring that the DMSO concentration was constant across the plate for all assays.

All data was normalized to the mean of 16 high and 16 low control wells on each plate. A four parameter curve fit was then applied.

### Alternative Method for S1P3 GTPγS Assay

S₁P₃ expressing RBL membranes (1.5 μg/well) were homogenised by passing through a 23G needle. These were then adhered to WGA-coated SPA beads (0.125 mg/well) in assay buffer (HEPES 20 mM, MgCl₂ 10 mM, NaCl 100 mM and pH adjusted to 7.4 using KOH 5M). GDP 10 μM FAC and saponin 90 μg/ml FAC were also added

After 30 minutes precoupling on ice, the bead and membrane suspension was dispensed into white Greiner polypropylene LV 384-well plates (5 μl/well), containing 0.1 μl of compound. 5 μl/well [³⁵S]-GTPγS (0.5 nM for S₁P₁ or 0.3 nM for S₁P₃ final radioligand concentration) made in assay buffer was then added to the plates. The final assay cocktail (10.1 μl) was then sealed, spun on a centrifuge, then read immediately on a Viewlux instrument.

Exemplified compounds had a pEC50<7, many had a pEC50<6. Examples 1 to 3, 7 to 10 and 12 to 13 had a pEC50<5.

1. A compound of formula (I) or a pharmaceutically acceptable salt thereof: 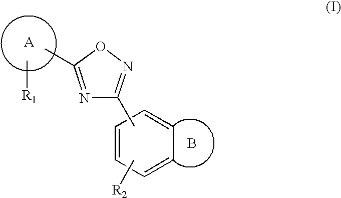
A is phenyl or a 5 or 6-membered heteroaryl ring;
R₁ is up to two substituents independently selected from halogen, C₍₁₋₃₎alkoxy, C₍₁₋₃₎fluoroalkyl, cyano, optionally substituted phenyl, C₍₁₋₃₎fluoroalkoxy, C₍₁₋₆₎alkyl and C₍₃₋₆₎cycloalkyl;
R₂ is hydrogen, halogen or C₍₁₋₄₎alkyl;
B is a 7 membered saturated ring selected from the following: 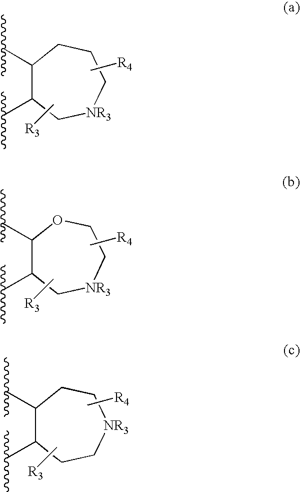
R₃ is hydrogen or C₍₁₋₃₎alkyl optionally substituted by oxygen;
R₄ is (CH₂)₁₋₃CONH₂, (CH₂)₁₋₃OH, CO₂H or (CH₂)₁₋₃CO₂H.

2. A compound of formula (I) or a pharmaceutically acceptable salt thereof, wherein:
A is phenyl or pyridyl;
R₁ is up to two substituents independently selected from chloro, cyano and isopropoxy;
R₂ is hydrogen;
B is (a) or (b);
R₃ is hydrogen;
R₄ is (CH₂)₂CONH₂, (CH₂)₁₋₃OH, CO₂H or (CH₂)₁₋₃CO₂H.

3. A compound of claim 1 selected from:
3-[7-(5-{3-chloro-4-[(1-methylethyl)oxy]phenyl}-1,2,4-oxadiazol-3-yl)-2,3,4,5-tetrahydro-1H-2-benzazepin-1-yl]propanamide;
3-[7-(5-{3-chloro-4-[(1-methylethyl)oxy]phenyl}-1,2,4-oxadiazol-3-yl)-2,3,4,5-tetrahydro-1H-2-benzazepin-1-yl]propanoic acid;
3-[7-(5-{3-chloro-4-[(1-methylethyl)oxy]phenyl}-1,2,4-oxadiazol-3-yl)-2,3,4,5-tetrahydro-1H-2-benzazepin-1-yl]-1-propanol;
[7-(5-{3-chloro-4-[(1-methylethyl)oxy]phenyl}-1,2,4-oxadiazol-3-yl)-2,3,4,5-tetrahydro-1,4-benzoxazepin-3-yl]methanol;
7-(5-{3-chloro-4-[(1-methylethyl)oxy]phenyl}-1,2,4-oxadiazol-3-yl)-2,3,4,5-tetrahydro-1,4-benzoxazepine-3-carboxylic acid;
3-[7-(5-{3-chloro-4-[(1-methylethyl)oxy]phenyl}-1,2,4-oxadiazol-3-yl)-2,3,4,5-tetrahydro-1,4-benzoxazepin-3-yl]propanoic acid;
[9-(5-{3-Chloro-4-[(1-methylethyl)oxy]phenyl}-1,2,4-oxadiazol-3-yl)-2,3,4,5-tetrahydro-1,4-benzoxazepin-5-yl]acetic acid;
[9-(5-{3-cyano-4-[(1-methylethyl)oxy]phenyl}-1,2,4-oxadiazol-3-yl)-2,3,4,5-tetrahydro-1,4-benzoxazepin-5-yl]acetic acid;
[9-(5-{5-Chloro-6-[(1-methylethyl)oxy]-3-pyridinyl}-1,2,4-oxadiazol-3-yl)-2,3,4,5-tetrahydro-1,4-benzoxazepin-5-yl]acetic acid;
4-[9-(5-{3-cyano-4-[(1-methylethyl)oxy]phenyl}-1,2,4-oxadiazol-3-yl)-2,3,4,5-tetrahydro-1,4-benzoxazepin-5-yl]butanoic acid;
4-[9-(5-{5-chloro-6-[(1-methylethyl)oxy]-3-pyridinyl}-1,2,4-oxadiazol-3-yl)-2,3,4,5-tetrahydro-1,4-benzoxazepin-5-yl]butanoic acid;
4-[9-(5-{3-chloro-4-[(1-methylethyl)oxy]phenyl}-1,2,4-oxadiazol-3-yl)-2,3,4,5-tetrahydro-1,4-benzoxazepin-5-yl]butanoic acid;
and pharmaceutically acceptable salts thereof.

4. (canceled)

5. (canceled)

6. (canceled)

7. (canceled)

8. (canceled)

9. (canceled)

10. A pharmaceutical composition comprising a compound according to claim 1.

11. A method of treatment for conditions or disorders in mammals including humans which can be mediated via the S1P1 receptors which comprises administering to the sufferer a therapeutically safe and effective amount of a compound of formula (I) or a pharmaceutically acceptable salt thereof.

12. A method of treatment according to claim 11, wherein the condition is lupus erythematosis.
